(19) United States
(12) Patent Application Publication
(10) Pub. No.: US 2019/0198172 A1
(43) Pub. Date: Jun. 27, 2019

(54) SYSTEMS, METHODS, AND DIAGNOSTIC SUPPORT TOOLS FOR FACILITATING THE DIAGNOSIS OF MEDICAL CONDITIONS

(71) Applicant: Robert P. Nelson, JR., Indianapolis, IN (US)

(72) Inventor: Robert P. Nelson, JR., Indianapolis, IN (US)

(21) Appl. No.: 16/327,711

(22) PCT Filed: Aug. 22, 2017
(86) PCT No.: PCT/US17/48080

§ 371 Date: Feb. 22, 2019

Related U.S. Application Data
(60) Provisional application No. 62/378,202, filed on Aug. 22, 2016.

(51) Int. Cl. G16H 50/20; G16H 50/30; G16H 10/20

(52) U.S. Cl. CPC G16H 50/20; G06Q 50/10; G16H 10/20; G16H 50/30

(57) ABSTRACT

Systems and methods for the accurate and efficient assimilation of clinical and laboratory findings to facilitate a medical diagnostic process are provided. Additionally, such systems and methods may also be employed to facilitate education with respect to specific medical conditions. The systems of the present disclosure comprise a network-based system configured to analyze user input in the form of phenotypic manifestations and, in some cases, pathognomonic data collected from a patient to provide a focused group of possible medical conditions that correlate therewith. Further, in certain embodiments, the systems can automatically generate a list of inquiries, based on the user input and non-discounted medical conditions, to facilitate an efficient inquiry process. Such systems may also be used for educational purpose to facilitate a user's understanding of the pathogenesis and phenotypic manifestations of medical conditions. Methods for using such system for diagnostic and/or educational purposes are also provided.

### PRIORITY

This application is related to and claims priority benefit of U.S. Provisional Patent Application Ser. No. 62/378,202 to Nelson filed Aug. 22, 2016. The entire content of the aforementioned priority application is hereby incorporated by reference in its entirety into this disclosure.

### BACKGROUND

The biomedical field of immunology has historically been linked with allergy (asthma, hay fever, eczema) but is currently emerging as a separate discipline. Even in recent history, common understanding of the human immune response was limited to the basic visualization of cells and measurement of cell products. In recent years, an understanding of the complexity of the science behind immunology has emerged, as well as how it is related to human illness.

Indeed, the field of immunology has seen tremendous growth and advancements with improvements in tools and technologies (e.g., cell-based assays, microarrays, mass spectrometry, etc.) that has informed a fresh understanding of the nature of the immune response and the translation of that knowledge into new methods of diagnosing and treating diseases. Recent advances in genomics and proteomics have also revolutionized our general understanding of biology in the past twenty-plus years. These advancements have helped elucidate the complex networks and interplay of cells, proteins and tissues of the immune system. For example, sequencing of the human and other model organism genomes has produced increasingly large volumes of data relevant to immunology research. Increasing amounts of laboratory data and genomic results reported in scientific literature are making their way into patient electronic medical records (EMR).

As such, the landscape of the relationship between human disease and immunology is changing rapidly. It is now commonly understood that immunodeficiency diseases and/or disorders are no longer limited to a small, easily-defined patient segment, but instead that immune responses are key to the development of many common disorders not traditionally viewed as immunological in nature including, without limitation, metabolic, cardiovascular, neurodegenerative, rheumatological and neoplastic diseases. Furthermore, overlap conditions are being described at a rapid pace. The practical effect of these recent changes is that many currently practicing clinicians have received minimal clinical training in immunology. Indeed, even the acronyms and nomenclature used in the field have undergone and continue to experience significant changes. Accordingly, while immunology has vast potential in the areas of diagnostics and treatments, it is difficult for medical professionals to maintain a comprehensive and current understanding of this complex and constantly evolving field.

Furthermore, the relevance of immunology spans virtually the entire human lifecycle and can be applicable across all medical specialties. In general, primary immunodeficiencies are a large group of different disorders caused when one or more components of the immune system (various cells and proteins) do not work properly. As a healthy immune system helps the body to prevent and mediate the consequences of infections by microorganisms (i.e. bacteria, viruses, and fungi), people with primary immunodeficiencies are more vulnerable than individuals with healthy immune systems. Indeed, susceptibility to common infections appears to be an increasingly important factor in the acquisition of infectious disease as opposed to microbial virulence and differential distribution of exposures. Accordingly, primary immunodeficiencies may manifest as infections other than respiratory (e.g., the common cold, ear infections, pneumonia), but may also manifest as arthritis, skin rashes, anemia, autoimmunity and inflammatory bowel conditions. Accordingly, not only is the landscape of immunodeficiency disorders rapidly changing, but the clinical presentation of primary immunodeficiency disorders can be extremely diverse and not limited to a particular patient subset.

Lack of exposure to or knowledge of disorders can limit a health care provider's ability to accurately and efficiently diagnose and/or treat a medical condition. The management of a patient with a primary immunodeficiency is entirely different than a patient with normal immune function. For example, in many cases, patients are typically diagnosed with and/or treated for immunodeficiencies only after they have been subjected to various other treatments and have failed to improve. Furthermore, the steps to be taken for the effective management of a patient often varies significantly between different diseases and disorders, whether immunodeficiency is a feature thereof or not.

A delay in accurate diagnosis and the initiation of effective treatment can be deleterious in any case, especially with respect to presence or absence of immunodeficiency. The paucity of trained immunologists makes it even more difficult to address this societal problem via traditional consultative and referral services. Accordingly, there is a need for an easy-to-use and accurate learning tool and diagnostic support tool to help educate healthcare providers and sift through the vast array of medical data that is currently available (including, for example “key indicators” of disease) in a meaningful manner. Furthermore, there is a significant need for a simple and reliable tool that allows medical professionals to effectively incorporate relevant and current immunoinformatics into their diagnostic and treatment approaches. Such resources will not only promise the quicker and accurate detection of disorders across medical specialties, but will provide an innovative mechanism to recognize and detect patients with primary immunodeficiencies.

### BRIEF SUMMARY

The systems, methods and techniques of the present disclosure comprise and utilize a tool for facilitating the differential diagnosis of immune and other disorders. The system may be delivered through an interactive network-based system using a software program (hosted or otherwise) in communication with a comprehensive medical reference database and is comprehensive, easy-to-use and readily updatable. This permits the provision of a high degree of accuracy. The system of the present disclosure can also provide an educational component to facilitate a user's expertise and exposure to various immunodeficiencies and other medical disorders or diseases; the key indicators therefore and the diagnostic tests applicable thereto.

In at least one exemplary embodiment, a method for detecting a medical condition in a subject is provided, such method comprising the steps of: (a) displaying a list of inquiries to a user, the list of inquiries formulated to distinguish between key indicators of a plurality of medical conditions and as compared to a healthy subject; (b) receiving, on a server, a set of data from a user, the set of data regarding a subject and in response to the list of inquiries; (c) executing a first application by a processor to reference the set of data received against a reference database and identify a subset of medical conditions pursuant to a first algorithm, the reference database comprising a plurality of medical conditions and associated key indicators and data associated with each medical condition, and the identified subset of medical conditions comprising medical conditions that correlate with the received set of data; (d) executing at least a second application by the processor to: generate an updated list of inquiries to distinguish between the medical conditions of the identified subset, and transmit the updated list to the user over the network; (e) receiving, on the server, a subsequent set of data from the user, the subsequent set of data in response to the updated list of inquiries; (0 repeating steps (c)-(e) unless and until the identified subset of medical conditions either consists of a manageable group of medical conditions or an updated list of inquiries cannot be generated due to lack of distinction between the key indicators and data of each medical condition of the identified subset; and referencing the subsequent set of data against the identified subset of medical conditions and, pursuant to a second algorithm executed by the processor, identifying medical conditions therein that correlate with the subsequent set of data received from the user.

The manageable group of medical conditions may comprise any number of medical conditions defined by an administrator or other user of the system; however, in at least one embodiment, a manageable group comprises one hundred or less medical conditions. In yet other embodiment(s), the manageable group of medical conditions comprises fifty or less medical conditions, seventy-five or less medical conditions, thirty or less medical conditions, fifteen or less medical conditions, or even one or zero medical conditions (where zero medical conditions may be indicative of the subject not experiencing an active condition of interest).

The medical conditions may comprise any general medicinal and/or pediatric condition. In at least one embodiment, the medical conditions are selected from a group consisting of conditions characterized by deficiency of immune function or regulation, autoimmune diseases, auto-inflammatory diseases, and infectious diseases. For example, the conditions characterized by deficiency of immune function may comprise primary immunodeficiency conditions or non-primary immune-mediated conditions. Additionally or alternatively, the auto-inflammatory diseases may comprise rheumatologic conditions.

The first algorithm utilized by the method may comprise a negative selection algorithm such that the step of executing a first application by a processor to reference the set of data against a reference database further comprises disregarding those medical conditions that do not correlate with the set of data. The set of data may comprise key indicator data related to the subject and, in at least one exemplary embodiment, the key indicator data comprises physical examination findings, laboratory results, and/or chromosomal analysis data.

The second algorithm may comprise a positive selection algorithm and the subsequent set of data received comprises pathognomonic data exhibited by the subject. The pathognomonic data may comprise one or more specific characteristics indicative of a medical condition which, when taken in conjunction with the already narrowed down subset, may be especially effective at identifying a likely diagnosis.

In certain embodiments of the method of the present disclosure, step (d) further comprises generating the updated list of inquiries based on distinctions identified by a third application between the key indicators and data associated with each medical condition of the identified subset. This may be performed automatically by the third application (for example, where the third application comprises a machine-learning service) or may performed manually by a user (administrator or otherwise). Where a machine-learning service is employed, the machine-learning service may analyze the reference database comprising the plurality of medical conditions and their associated key indicators and data using a statistical analysis methodology. For example, the machine-learning service may employ decision tree learning, inductive logic programming, similarity metric learning, clustering, and/or Bayesian network analysis.

Methods hereof may additionally comprise step of executing a fourth application by the processor to recommend one or more diagnostic tests, the results of which may be useful in distinguishing between the medical conditions of the identified subset. Additionally or alternatively, the present methods may further comprise the step of performing a diagnostic test on the subject, wherein the subsequent set of data comprises results of the diagnostic test. In this manner, the methods of the present disclosure can further facilitate the performance of the most-effective laboratory tests in furtherance of the data that has already been collected and analyzed, and, likewise, reduce waste and emotional stress on the subject.

In at least one embodiment, the method further comprises the steps of: receiving, on the server, a request from the user to schedule a diagnostic test with a laboratory; and executing an application by the processor to submit a request, over the network, to the laboratory to schedule the diagnostic test. Furthermore, the method may further comprise the step of transmitting a confirmation of the scheduled diagnostic test to the user over the network. Still further, the method may further comprise the step of treating the subject for a diagnosed medical condition selected from the identified subset of medical conditions (such treatments as may be now known in the art or hereinafter developed in connection with the relevant diagnosis—for example, such as administering pharmaceuticals, surgery, lifestyle changes, etc.).

Interactive diagnostic support systems are also provided in the present disclosure. In at least one embodiment, such systems comprise a platform comprising a processor and memory, both of which are coupled with at least one server. The at least one server may be in operative communication with a network and accessible by at least one user via one or more clients. The server may also comprise at least one application executable by the processor and be configured to interact with data stored at least partially within the memory of the platform.

In at least one exemplary embodiment, the platform of the system is configured to display (via a user interface or otherwise) a list of inquiries for distinguishing between a plurality of medical conditions, receive (on the server, for example) data from a user in response to the list of inquiries, access and compare the received data from the user with medical reference data stored at least partially within the memory of the platform to identify a subset of medical conditions that correlate with the received data, generate an updated list of inquiries to distinguish between the medical conditions of the identified subset, and display (via a user interface, for example) the subset of medical conditions and the updated list of inquiries. In at least one embodiment, the received data is associated with a patient and comprises key indicators and, where desired, pathognomonic data associated with the patient. In at least one exemplary embodiment, the medical conditions of the system are selected from a group consisting of conditions characterized by deficiency of immune function or regulation, autoimmune diseases, auto-inflammatory diseases, and infectious diseases.

The platform may additionally be configured to identify and display one or more diagnostic tests, the results of which would be useful in distinguishing between the medical conditions of the identified subset. For example, in at least one embodiment, the platform may be configured to execute one or more applications to identify unknown variables associated with the medical conditions within the then-current subset of medical conditions, as well as identify patterns in such unknowns (for example, and without limitation, where the answer to a single unknown may eliminate multiple medical conditions from the subset or where a positive answer to a single unknown may positively correlate with one or more medical conditions).

The server of the platform may be in operative communication with one or more laboratories of the network. There, the platform may be configured to interact with the one or more laboratories (or their respective systems—online, intranet, or otherwise) in response to a request from the user to schedule a diagnostic test. Accordingly, the platform can automatically reach out and schedule a diagnostic test with one or more third-party/external laboratories pursuant to user input received within the system of the present disclosure.

The system may also comprise a reference database comprising a plurality of medical conditions, where the application of the system is configured to interact with the data stored within the reference database. Such medical reference data may comprise a plurality of medical conditions, with one or more phenotypic manifestations, characteristics, molecular causes, and categories assigned to each medical condition. The medical reference data may be stored at least partially within the memory of the platform and, in at least one exemplary embodiment, may be updatable in real-time via multiple users over the network. Additionally or alternatively, the medical reference database may be in communication with and/or further comprise one or more databases that are external to the system (maintained by third-parties or otherwise). For example, the medical reference database may be linked to and/or otherwise in communication with the HUGO Gene Nomenclature Committee database of human gene nomenclature and the data stored therein.

As previously noted, the platform of the interactive diagnostic support system may be further configured to display via the user interface one or more data sets identified by a user, wherein each data set comprises information on a medical condition. The information on a medical condition may comprise at least a key indicator or pathognomonic data indicative of a subject experiencing one or more medical conditions.

Yet other embodiments of the interactive support system hereof may be geared towards educational purposes. There, the platform may be configured as previously described; however, in such embodiments, the platform is configured to display via a user interface a list of available data sets, each data set associated with a medical condition, receive (on the server) input from a user related to a first data set selected from the list of available data sets, display via the user interface the first data set to the user; wherein the first data set comprises information on the medical condition associated with the first data set. Like previously described embodiments, the information on the medical condition may comprise at least a key indicator or pathognomonic data indicative of a subject experiencing one or more medical conditions. Furthermore, in the educational and diagnostic embodiments of the system, the information of the first data set may further comprise information on the medical condition associated with the first data set from a scientific journal, text book, encyclopedia, patient case report, or a scientific community listerv.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 shows a schematic/block diagram of an interactive diagnostic and educational support system according to an exemplary embodiment of the present disclosure;

FIGS. 2-7A show exemplary embodiments of user interfaces that may be used with the interactive diagnostic and educational support system of FIG. 1 according to exemplary embodiments of the present disclosure;

FIG. 7B shows a flow chart representing a method for detecting a subject with a medical condition using the interactive diagnostic and educational support system of FIG. 1 and/or according to exemplary embodiments of the present disclosure; and

FIGS. 8-12 show exemplary embodiments of additional user interfaces that may be used with the interactive diagnostic and educational support system of FIG. 1, according to exemplary embodiments of the present disclosure.

The disclosed embodiments and other features, advantages, and disclosures contained herein, and the matter of attaining them, will become apparent and the present disclosure will be better understood when the following description is taken in conjunction with the accompanying drawings/figures. As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended, with any additional alterations and modifications and further applications of the principles of this disclosure being contemplated hereby as would normally occur to one skilled in the art. On the contrary, it is intended to cover alternatives, modifications, and equivalents as may be included within the spirit and scope of this application as defined by the appended claims. While this technology may be illustrated and described in one or more preferred embodiments, the devices, systems, and methods hereof may comprise many different configurations, forms, materials, and accessories.

For example, the systems, methods and techniques of the present disclosure will be described in the context of a tool for providing the differential diagnosis of immune and other disorders. The system may be delivered through an interactive network-based system using a software program (hosted or otherwise) in communication with a comprehensive medical reference database and is comprehensive, easy-to-use and readily updatable. This permits the provision of a high degree of accuracy. The system of the present disclosure can also provide an educational component to facilitate a user's expertise and exposure to various immunodeficiencies and other medical disorders or diseases; the key indicators therefore and the diagnostic tests applicable thereto. Ultimately, the systems and methods hereof may be used by medical professionals and others to facilitate the prompt and accurate recognition and detection of conditions in subjects. While the systems, methods, and techniques of the present disclosure apply in a wide variety of contexts, including, but not limited to, diagnostic support tools and methods for the diagnosis of, or education regarding any medical condition, in at least one exemplary embodiment, the systems, methods, and techniques of the present disclosure can be geared towards immunodeficiencies. There, use of the inventive concepts hereof permit the timely recognition and detection of subjects with primary immunodeficiencies and furthermore facilitate elucidation of human immunological function.

In the following description, numerous specific details are set forth in order to provide a thorough understanding of the present disclosure. Particular examples may be implemented without some or all of these specific details. In other instances, well known process operations and/or system configurations have not been described in detail to not unnecessarily obscure the present disclosure.

Various techniques and mechanisms of the present disclosure will sometimes describe a connection between two components. Words such as attached, affixed, coupled, connected, and similar terms with their inflectional morphemes are used interchangeably, unless the difference is noted or made otherwise clear from the context. These words and expressions do not necessarily signify direct connections, but include connections through intermediate components and devices. It should be noted that a connection between two components does not necessarily mean a direct, unimpeded connection, as a variety of other components may reside between the two components of note. For example, a workstation may be in communication with a server, but it will be appreciated that a variety of bridges and controllers may reside between the workstation and the server. Consequently, a connection does not necessarily mean a direct, unimpeded connection unless otherwise noted.

The detailed descriptions which follow are presented in part in terms of algorithms and symbolic representations of operations on data bits within a computer memory representing alphanumeric characters or other information. A computer generally includes a processor for executing instructions and memory for storing instructions and data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be kept in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms commonly associated with mental operations performed by a human operator (such as “comparing”). No such capability of a human operator is necessary, or desirable in most cases, in any of the operations described herein which form part of the embodiments of the present application; the operations are machine operations. Indeed, a human operator could not perform many of the machine operations described herein due to the networking and vast distribution capabilities of the present disclosure. This is especially true with respect to the machine-learning services that provide ranking, clustering, classifying, data aggregation, and prediction techniques.

Useful machines for performing the operations of one or more embodiments hereof include general purpose digital computers, microprocessors, tablets, handheld or otherwise mobile devices, or other similar devices. In all cases the distinction between the method operations in operating a computer and the method of computation itself should be recognized. One or more embodiments of the present disclosure relate to methods and apparatus for operating a computer in processing electrical or other (e.g., mechanical or chemical) physical signals to generate other desired physical manifestations or signals. The computer and systems described herein operate on one or more software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level of coding of the instructions that is interpreted to obtain the actual computer code. The software module may also include a hardware component, wherein some aspects of the algorithm are performed by the circuitry itself rather as a result of an instruction.

In the following description, several terms which are used frequently have specialized meanings in the present context. A “subject” or “patient” as used herein are interchangeable and refer to a mammal, preferably a human, that is being evaluated for a medical condition.

The terms “immune disorder,” “primary immunodeficiency,” “immunodeficiency disease” and “immunodeficiency condition” are used interchangeably herein and mean a group of disorders and diseases characterized by deficiencies of the immune system function and/or regulation.

The term “key indicator,” as used herein, means an observable qualitative or quantitative characteristic that, when taken in combination with other key indicators, permits the separation of pathological disease states from normal. For example, and without limitation, key indicators may comprise a phenotypic manifestation of a disease, historical presence or absence of illness, physical signs, and various combinations of standard screening clinical laboratory test results from a patient.

A “machine-learning service” or “machine-learning” is a software application running on the platform or system of the present disclosure that provides the necessary functionality for one or more software applications to learn from interactions with the users and/or a medical reference database or other databases of, or accessible by, the system hereof.

The term “phenotypic manifestation” as used herein means an observable physical or biochemical characteristic of a subject including, without limitation, environmental influences, genetic makeup, the expression of a specific trait or symptom, the presence of a specific pathogen within a biological sample collected from the subject, the presence of characteristic inflammatory lesions described and reported with pathological specimen, and the like.

The terms “network,” “local area network,” “LAN,” “wide area network,” or “WAN” mean two or more computers which are connected in such a manner that messages may be transmitted between the computers. In such computer networks, typically one or more computers operate as a “server,” which runs one or more applications capable of accepting requests from clients and giving responses accordingly. Servers can run on any computer including dedicated computers, which individually are also often referred to as “the server” and typically comprise—or have access to—large storage devices or storage environments (such as, for example, hard drives, virtual databases, and/or the like) and communication hardware to operate peripheral devices such as printers or modems. Servers can also be configured for cloud computing, which is Internet-based computing where groups of remote servers are networked to allow for centralized data storage. Such cloud computing systems enable users to obtain online access to computer services and/or resources.

Other computers, termed “workstations” or “clients” provide access to one or more interfaces so that users of computer networks can access the network resources, such as shared data files, via common peripheral devices and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program or application that carries out one or more specific functions and may be used alone or combined with other modules of the same system or program.

The term “Browser” refers to a program which is not necessarily apparent to the user, but which is responsible for transmitting messages between a workstation and the network server and for displaying and interacting with the network user. Browsers are designed to utilize a communications protocol for transmission of text and graphic information over a worldwide network of computers, namely the “World Wide Web” or simply the “Web.” Examples of Browsers compatible with one or more embodiments described in the present disclosure include, but are not limited to, the Chrome browser program developed by Google Inc. of Mountain View, Calif. (Chrome is a trademark of Google Inc.), the Safari browser program developed by Apple Inc. of Cupertino, Calif. (Safari is a registered trademark of Apple Inc.), Internet Explorer program developed by Microsoft Corporation (Internet Explorer is a trademark of Microsoft Corporation), the Opera browser program created by Opera Software ASA, the Firefox browser program distributed by the Mozilla Foundation (Firefox is a registered trademark of the Mozilla Foundation), or any other Browsers or like programs currently in use or hereinafter developed. Although the following description details operations in terms of a graphic user interface of a Browser, it will be understood that one or more embodiments disclosed in the present disclosure may be practiced with text based interfaces, voice or visually activated interfaces, mobile application interfaces, or any other interfaces now or hereinafter developed that have some or many of the functions of a graphic-based Browser.

Overview:

To promote a thorough understanding of the present application, a brief overview of the functionality associated with the disclosed systems and methods will first be provided, followed by a more detailed description of the underlying componentry and steps of implementation. While much of the description herein focuses on immunodeficiencies and immunology, it will be appreciated that this is presented for clarity of explanation alone and in no way limits application of the present disclosure. Like immunology, other areas of medicine and medicinal specialties are constantly evolving and/or involve a cumbersome breadth of data that needs to be accessible to healthcare providers in a meaningful manner such that it enables access to a diagnostic support tool enabling quick and accurate diagnosis. The inventive concepts of the present disclosure may be applied to any category of disease or disorder that can be identified through the categorization and comparison of symptoms and key indicators and/or where it may be desirable to quickly navigate a large amount of aggregated key indicator data.

Primary immunodeficiencies are inherently difficult to diagnose as they are characterized in many different ways, are not limited to a particular class of subjects (i.e. age, sex, etc.), and can be associated with a variety of clinical presentations. Heretofore medical professionals and others have had to rely on their own knowledge and trial and error in diagnosing primary immunodeficiency diseases; however, it is extremely difficult for such individuals to gain (and keep) a comprehensive and current understanding of the rapidly evolving field of immunology such that they can make informed decisions.

Under conventional structures, the prompt incorporation of new research findings into routine practice necessitates regular reading, evaluation, and integration of the current knowledge gathered either from personal experience or published literature. As most medical professionals do not specialize in more than one medical specialty, most medical fields, such as immunology, are typically secondary to medical professional's area of practice and, thus, not a primary focus. As such, personal practice is unlikely to enable a practicing healthcare provider to gain a well-rounded overview of current findings in those fields where his or her practice is not focused (e.g., immunological findings). Additionally, given the dramatic increase in studies and scientific articles published in various specialties of medicine, depending on published literature is impractical—if not impossible—and, while the information may be available, there is not a conventional system or method for its incorporation into clinical decision-making across the board.

In general, the present disclosure provides novel systems and methods for the assimilation of clinical and laboratory findings to facilitate the diagnostic process. Perhaps more specifically, the systems and methods hereof are configured to provide a series of comprehensive clinical and laboratory-based inquiries to collect data points related thereto from a subject. Such collected data is then referenced in real-time against one or more medical reference databases pursuant to one or more algorithms for the purpose of identifying increasingly narrow subsets of medical conditions that are consistent with the subject's symptoms and key indicators. Ideally, the process will continue until the list of possible medical conditions narrows to a manageable group, such as one hundred (100) or fewer medical conditions, seventy-five (75) or fewer medical conditions, fifty (50) or fewer medical conditions, thirty (30) or fewer medical conditions, or fifteen (15) or fewer medical conditions, for example, or any other number of medical conditions as may be programmed to equate with a manageable group. Further, in at least one embodiment, the process continues until the list of likely medical conditions narrows to a single condition that is indicative of a patient's diagnosis. Thereafter, in at least one exemplary embodiment, the process may also identify a possible personalized intervention for the patient at issue based on the resulting diagnosis and a healthcare provider may subsequently treat such subject.

Where the process is unable to identify increasingly narrow subsets of medical conditions down to a manageable group, the results may be indicative of the subject not clinically experiencing a medical condition for which the system and/or method is testing. For example, where the system and/or method is directed toward immunology, if the system is unable to narrow a subset down to a manageable group of medical conditions based on a user's entry of data points from the subject, either more data may be required or the subject may simply not be experiencing an immune-derived condition.

In at least one exemplary embodiment, the inquiries presented to the user may be dynamic in nature. In other words, the content and/or subject of subsequent inquires may be dependent on the data previously collected from the user and/or the currently identified subset of potential conditions. For example, when the user (i.e., a medical professional, other health care provider, medical student or other individual) accesses the system, a diagnostic tool of the system provides a list of inquiries relating to key indicators for various deficiencies. Where the system is not programmed to be specific to a particular medical specialty, this initial list of inquiries may be general and/or designed to preliminarily classify a patient's deficiency within a smaller subset of medical specialties before subsequently presenting specialty-specific inquiries. Alternatively, where the system is programmed to be specific to a particular medical specialty (such as immunology, for example), the initial list of inquiries may comprise more specialty related content.

In response to the initial list of inquiries, the user inputs a first set of data into the system and the tool references such first series of input data against the medical reference database pursuant to a defined algorithm (e.g., a negative selection algorithm) to generate a first subset of potential medical conditions that aligns with the collected data. Thereafter, unless the first subset of potential medical conditions is limited to only a single medical condition, the tool compiles and presents a second list of inquiries to the user, such list based on the previously collected data and the data associated with the first subset of potential medical conditions. In at least one exemplary embodiment, the second list of inquiries is automatically compiled by the system. After inputting a second set of data into the system in response to the second list of inquiries, the tool references the second set of data against the medical database and the first subset of potential medical conditions to generate a second subset of potential medical conditions that comprises comparatively fewer medical conditions than the first subset.

This process is repeated with respect to the data in the medical reference database associated with the first subset of potential medical conditions (i.e. the user is presented with another list of inquiries, enters the data, and the data is referenced against the data in the database associated with the first subset of potential medical conditions) to generate progressively narrow subsets of potential medical conditions pursuant to a defined algorithm (e.g., a positive or negative selection algorithm) until either all of the programmed inquiries have been presented to the user or, of the number of deficiencies, disorders, or diseases within the reference database(s) (e.g., where the system is geared towards use with immunological disorders, currently approximately 340 molecularly defined primary immunodeficiencies), the list is reduced to a manageable and informed list of potential medical conditions (i.e. one hundred or less conditions, fifteen or less conditions, etc.). In at least one exemplary embodiment, when the system and/or method identifies a manageable group of medical conditions, a subsequent list of inquiries is displayed to the user (populated automatically or otherwise), with such list of inquiries directed towards acquiring pathognomic data or the like from the subject. As such data is typically extremely specific to particular medical conditions, the system and method may then employ a positive selection algorithm to identify which medical conditions of the manageable group are positive for the input data. In this manner, the system facilitates the recognition, categorization, and sorting of all key indicators by their prevalence in diseases and/or disorders, as well as their incisiveness.

Additionally, in at least one embodiment, the tool may also identify and/or suggest, based on previously entered data, additional data sets and/or test results (e.g., laboratory tests) that may be beneficial in identifying and/or confirming the underlying medical condition. The system may automatically identify such information by analyzing/mapping the data in the medical reference database associated with the current subset of potential medical conditions and identifying one or more patterns of data points therein that may further narrow the results. If such a pattern is detected, then system can be programmed to indicate what type(s) of data sets and/or test results may be useful in further narrow the current subset of potential medical conditions.

A major advantage of the systems and methods provided herein are that they provide simple avenues for students to learn about different medical specialties, which is especially useful in the rapidly-expanding field of primary immunodeficiencies and their molecular causes. Indeed, using the systems and methods of the present disclosure, a user may easily access clear and current information (embodiments of which may be updated anywhere from real-time as soon as new data is available or on a particular schedule (e.g., monthly) as compared to years for standard textbook revisions) on a variety of disorders (e.g., immunodeficiencies, hematology disorders, cancer, rheumatologic conditions, etc.). Such systems and methods can also provide a diagnostic support tool for medical students, residents, physicians, researchers, and other health care providers that assimilates up-to-date clinical and laboratory findings into a real-time algorithm that facilitates the speed and accuracy of the diagnostic process.

Particular embodiments of the systems and methods hereof provide time-saving and seamless connectivity to location-specific laboratories and other testing resources. Use of these comprehensive and interactive systems and methods can significantly improve the recognition and detection of patients with primary immunodeficiencies and facilitate the elucidation of immunology. Certain exemplary embodiments of the system may even be in operative communication (over a network or otherwise) with one or more laboratories such that a user may submit a request directly to a desired laboratory to schedule an analysis and/or communicate directly therewith. For example, where additional data may be useful with regards to a particular disease (either because it has been recommended by the tool or otherwise identified as appropriate by the healthcare provider), the user may easily submit a request through the system to a laboratory of interest to schedule the analysis. Due to the network-based infrastructure of the tools and systems of the present disclosure, the laboratories available through the tool need not be limited to a single geographic area and may include laboratories throughout the United States of America or even the world.

System and Service Architecture:

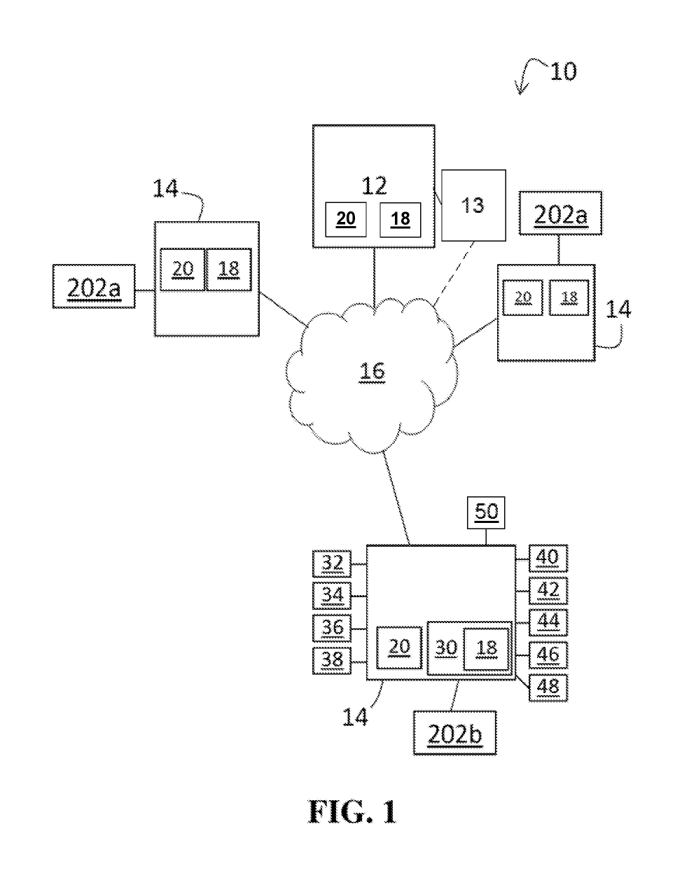

Now referring to the system and service architecture of the present disclosure, FIG. 1 is a high-level block diagram of a computing environment through which aspects of the presently disclosed system and methods may be implemented. As shown in FIG. 1, in at least one embodiment, the education and diagnostic support system 10 of the present disclosure comprises at least one server 12, a database 13, and at least two clients 14 connected by a network 16. One or more users 202, such as healthcare providers described in further detail herein, can access the system 10 via the one or more clients 14. Specifically, in at least one embodiment, the education and diagnostic support system 10 is configured such that one or more users 202 can access the particular functionality of and/or data stored within the server 12/database 13 via a user interface (not shown) and the network 16. For example and without limitation, the network 16 may be operatively coupled with clients 14 via the Internet, an intranet (e.g., available over a hospital or university intranet), or any other connection. Accordingly, the system 10 is not limited by the geographical location of a user 202.

The computing environment may be configured similarly to a multi-user site in that numerous parties may register and/or access the server 12 via multiple—and commonly remote—clients 14. The server 12 is operatively coupled with the clients 14 over a network 16 or networking infrastructure and operates to run various applications 18 and store and/or access data stored either on the server 12 or accessible thereby as is known in the art. As previously mentioned, the database 13 may be local to a server 12 or separate therefrom (albeit accessible thereby). The server 12 may also comprise one or more applications 18 executable by one or more processors 20 of the server 12 (or as is otherwise known in the art). In at least one embodiment, the functionality of the present system 10 is provided to users 202 via a software as a service (SaaS) platform, such that the applications 18 are all run in the cloud and accessible by users 202 via the network 16. It will be appreciated, however, that one or more of the applications 18 of the system 10 may be run locally on the clients 14, on the server(s) 12, in the cloud, and/or in any other configuration or combination thereof that may be desired to optimally achieve the goals of the end user.

Furthermore, while only three clients 14 are shown in FIG. 1, this is only to simplify and clarify the description and no limitation is intended. Embodiments of the computing environment may have any number of clients 14 connected to the network 16, for example one, thousands, or even millions of clients 14. Likewise, while only one server 12 is depicted in FIG. 1, the computing environment may comprise a plurality of servers 12 (including, without limitation, compute and storage resources, which may be virtual, physical, or any combination thereof). It will also be understood that database 13 may comprise any database now known in the art or hereinafter developed, any number of individual databases, and, in at least one exemplary embodiment, database 13 may comprise a database server and/or a Deficiencies Module of the system 10 (described in additional detail herein). Additionally or alternatively, the database 13 may comprise a database 13 on a server 12 and need not be separate from the server(s) 12 at all. In any event, the data stored within the database 13 is accessible by one or more of the servers 12 (either directly or through the network 16) and comprises an updatable reference database comprising a plurality of medical conditions (e.g., immunodeficiency disorders) and the key indicators, pathognomonic data, and other information associated therewith. In at least one embodiment, at least one of the databases 13 includes data regarding potential tests or diagnostics that can be run, the resulting test results achieved therefrom, and/or information related to one or more laboratories and other testing resources. Additionally, as is known in the art, the servers 12 and/or clients 14 may comprise processors and other hardware (collectively represented as processor 20 in FIG. 1) to execute and run the various applications and/or perform the functionality described herein as may be appropriate.

The clients 14 may each comprise one or more network-accessible devices capable of executing one or more applications and/or accessing a web-based system through a Browser. A client 14 may be any type of workstation such as, for example, any type of computer, computing device, or system of a type known in the art such as a personal computer, mainframe computer, workstation, notebook, tablet or laptop computer or device, PDA, mobile telephone, smartphone or device, wearable, or any other computing or communications device having network interfaces (wireless or otherwise).

Users 202 may operate software 18 on one or more clients 14 (stored on a storage medium 30, such as a hard disk, flash memory, a solid-state drive, random access memory, etc., and executed by one or more processors 20)—such as a mobile application designed for use with a smartphone, wearable, or other mobile device—to both send and receive messages and/or data over the network 16 via server 12 and any of its associated communications equipment and software (not shown). Further, and as noted above, clients 14 may each comprise hardware and componentry as would occur to one of skill in the art such as, for example, one or more microprocessors (exemplary processors 20), memory (an exemplary storage medium 30), input/output devices (as noted below), device controllers, and the like. Clients 14 may also comprise one or more input devices that are operable by a user 202 such as, for example, a keyboard 32, keypad 34, pointing device 36, mouse 38, touchpad 40, touchscreen 42, microphone 44, camera 46, and/or any other data entry means (referred to as inputs 48), or combination thereof, known in the art or hereinafter developed, as well as visual and/or audio display means 50 for displaying or emitting output (e.g., a CRT or LCD display).

As shown in FIG. 1, each client 14 is connected to, and/or in communication with, the server 12 via a network 16. The network 16, which provides access to the education and diagnostic support system 10 and the functionality thereof, comprises any means for electronically interconnecting the server 12 and a client 14. In at least one exemplary embodiment, the network 16 comprises the Internet, a global computer network. Alternatively, the network 16 may be selected from a variety of different networks and/or cables including, but not limited to, a commercial telephone network, one or more local area networks, one or more wide area networks, one or more wireless communications networks, coaxial cable(s), fiber optic cable(s), and/or twisted-pair cable(s). Additionally, the network 16 may comprise equivalents of any of the aforementioned, or combinations of two or more types of networks and/or cables.

Furthermore, in at least one embodiment, the server 12 and a client 14 comprise a single computing device operable to perform the functions delegated to both server 12 and a client 14 according to the present disclosure. There, the network 16 may comprise the hardware and software means interconnecting the server 12 and client 14 within the single computing device. Accordingly, the network 16 may comprise packet-switched facilities (such as the Internet), circuit-switched facilities (such as the public-switched telephone network), radio-based facilities (such as a wireless network), or any other facilities capable of interconnecting a client 14 with the server 12.

It will be appreciated that where the computing environment comprises a plurality of clients 14, such clients 14 need not all comprise the same type of client 14 or be in communication with the network 16 and/or server 12 via the same type of communication link. As such, the computing environment may comprise some clients 14 configured to connect to/communicate with the server 12 via the Internet, for example, while other clients 14 are connected to the server 12 via a wired connection (e.g., a cable).

The education and diagnostic support system 10 may be implemented through any appropriate application architecture pattern now known or hereinafter developed. In at least one exemplary embodiment, the education and diagnostic support system 10 is delivered through an n-tier architecture in which presentation, application/business logic, and data management functions are logically and/or physically separated. This application architecture pattern provides benefits in the way of increased availability of the system to its users (i.e. reduced downtime), the minimization of the impact of any component failure, and the facilitation of disaster recovery. Additionally, third party applications (e.g., a third-party payment processor, third party laboratory networks, websites, and/or scheduling applications) may be interfaced with the system to system users additional functionality without sacrificing data security as such third-party applications need not be in direct communication with the data structures of the system.

System Operation and Users:

The education and diagnostic support system 10 supports at least two categories of users 202—healthcare provider users 202a and administrator users 202b—that can logon and access the system 10 via the client(s) 14. The healthcare provider users 202a may comprise any individual or entity that desires to utilize the education and diagnostic support system 10 in connection with learning about disorders/diseases (immunodeficiencies, for example) and/or diagnosing a medical condition. For example, a healthcare provider user 202a may comprise a student (medical or otherwise), resident, physician, researcher, laboratory, and like individuals or entities that may need access to a medical diagnostic tool.

An administrator user 202b may be any individual or business entity that plays an operational or governance role of the system 10. In at least one embodiment, the system 10 is operated by (or under the control of) one or more administrators. The administrators may be individuals, educational institutions, institutions of higher learning, healthcare provider networks, hospitals, hospital networks, health insurance provider networks, and/or representatives of the foregoing. An administrator user 202b may have broad security credentials and access permission that provide it with access to data stored throughout the system 10 (or limited portions thereof); rights to customize components, functionality and/or feature sets of the system 10 itself; the ability to run and view data analytics and/or patient records based on healthcare provider user 202a activity; and the authority to terminate or suspend a user's 202a account. Furthermore, with the appropriate credentials, an administrator user 202b may add to or update the reference database 13 of the system 10.

For example, there may be a system-wide administrator 202b that has the broadest security credentials and can manage the content of the reference database 13 and all other users 202a/b and components of the system 10. Likewise, there may be one or more other administrators 202b associated with a particular hospital network or university that has implemented the system 10 with its healthcare providers or students (respectively) (each a “client administrator 202b”). In such cases, the client administrators 202b may have security credentials such that they can manage and/or customize the system components and/or functionality accessible by its associated users 202a, but may not have rights to update or otherwise modify the reference database 13. For the avoidance of doubt, where the term “user 202” is used herein, it shall mean and include both categories of healthcare provider users 202a and administrative users 202b.

The available functionality of the education and diagnostic support system 10 varies depending on the category of user 202, considering that the different types of users 202 have different goals for accessing and utilizing the system 10. However, in all cases, the users 202 interact with the system 10 through one or more user interfaces to input information or access the functionality of the system 10 and/or data stored within the server 12, and such interfaces may comprise any configuration or design that is appropriate to achieve such purposes.

In at least one exemplary embodiment, the education and diagnostic support system 10 is delivered as an open platform environment, where anyone with access to the Internet may register as a healthcare provider user 202a thereof. For example, users 202a can gain access to the system 10 and underlying computing environment via a secure login interface as is commonly known in the art (e.g., creating an account, establishing a username and password, etc.). Alternatively, the system 10 may be delivered via a secure portal application. Accordingly, by entering a publicly-available website or a secure portal, a user 202a can register and gain access to the functionality provided by the education and diagnostic support system 10 (noting, however, that it may be desirable to employ a verification process in the event a healthcare provider 202a opens a new account or desires to be associated with an existing administrator account).

Still further, in at least one exemplary embodiment of the education and diagnostic support system 10, users 202 access the system 10 via a user-facing mobile application and/or widget designed to run on smartphones, tablets, tablet computers, wearables, and the like. It will be appreciated that such applications/widgets may be offered on both iOS and Android platforms, or in connection with any other mobile operating systems that are now known or hereinafter developed.

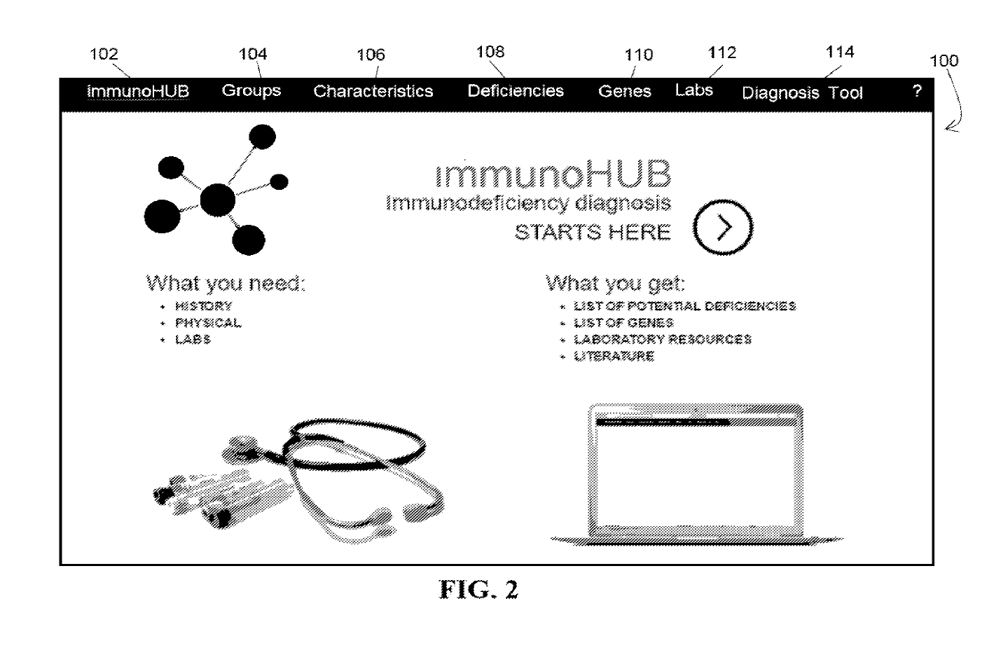

Now referring to FIGS. 2-7, examples of user interfaces (shown as viewed through a Browser) that may be used in connection with the system 10 are shown. Such interfaces are one way through which a user 202 may access and use the functionality of the education and diagnostic support system 10. Perhaps more specifically, a healthcare provider user 202a can login to the system 10 to view the educational materials therein, utilize the diagnostic tool thereof (that has access to the reference database 13), and/or schedule laboratory test(s) for a patient. Additionally or alternatively, an administrator user 202b can login to the system 10 to manage its associated healthcare provider users 202a and/or access or view real-time data trends.

It will be appreciated that while the examples of user interfaces provided herein comprise specific fields, dropdown menus, buttons and other graphical control elements, the user interfaces of the education and diagnostic support system 10 may be configured in any manner desired, customized pursuant to the particular functionalities provided by the system 10, and/or to request various types of information as appropriate from the various users 202 in light of their intended use of the system 10. Indeed, the embodiments illustrated in FIGS. 2-7 are provided merely by way of explanatory example and are not intended to be limiting in any way.

In an exemplary embodiment of the education and diagnostic support system 10, an individual must provide certain registration information and create an account as a condition precedent to accessing the system 10. The required information may include, without limitation, the potential user's name, address, other contact information, and/or any institution/hospital affiliation that might influence his or her use of the system 10. Once a user 202 has established a user account and, optionally, been assigned credentials, the user 202 may access the system 10. In at least one embodiment, upon logging into the system 10 a user 202 is directed to a homepage. FIG. 2 illustrates an example of a homepage interface 100 configured such that the user 202 can easily access the functionality of the system 10. For example, homepage interface 100 may comprise tabs (or other links) that link the user 202 to various modules of the system 10—i.e. a Groups Module (Group tab 104), a Characteristics Module (Characteristics tab 106), a Deficiencies Module (Deficiencies tab 108), a Genes Module (Genes tab 110), a Laboratories Module (Laboratories tab 112), and a Diagnosis Support Tool Module (Diagnosis Support Tool tab 114). To further illustrate the benefits of the systems and methods of the present disclosure, each of these Modules will now be described in additional detail.

Given the rapid pace of discovery associated with primary immunodeficiency disorders in particular, the principle challenge to a student or healthcare provider in the modern era is identifying all relevant data and establishing an accurate diagnosis quickly. Decreasing the time it takes to achieve an accurate diagnosis is a critical factor to achieving optimal patient care. For example, the medical management of a patient experiencing a primary immunodeficiency differs significantly from persons with normal immunological function and, as such, a misdiagnosis can be costly not only in terms of the financial cost of treatment and disease management, but also with respect to patient health and emotional outlook. Once a primary immunodeficiency is suspected, an optimal choice of care (if feasible) is efficient referral to a center that has particular expertise and experience in the diagnosis and management of patients with primary immunodeficiencies, which is significantly divergent from the optimal choice of care when a primary immunodeficiency is not at play. The Diagnosis Support Tool Module of the present disclosure addresses this issue; allowing for an increase in overall tempo of the diagnostic process, providing a high level of accuracy, and allowing for quick access to timely research and developments in the complex and rapidly evolving field of immunology as well as other medical fields, if desired. Indeed, the Diagnosis Support Tool Module of the system 10 provides a simple and automated process through which a user 202 provides patient-specific input/data and such data is referenced and correlated against a proprietary medical reference database 13 to identify one or more possible diagnoses (i.e. a subset of potential medical conditions).

As previously described, the steps of requesting patient data, referencing the data received against the medical reference database 13, and identifying a subset of medical conditions are repeated until, ideally, of the total number of molecularly defined primary immunodeficiencies, a manageable and informed list of potential medical conditions is identified. Thereafter, positive selection may be utilized (pursuant to an algorithm executed by the system, for example), to further narrow the manageable and informed list based on pathognomonic data from the subject or the like. The Diagnosis Support Tool Module is a comprehensive diagnostic support tool that can be extremely beneficial, especially in the field of immunology, as it can quickly and accurately provide a manageable subset of potential diagnoses or, ideally, a single, differential diagnosis. While in certain embodiments the Diagnostic Support Tool Module may be designed to narrow down the number of possible diagnoses as much as possible to arrive at a single diagnosis, in at least one exemplary embodiment, the Diagnostic Support Tool Module is more of a support tool than a diagnostic tool. In this at least one exemplary embodiment, the Diagnostic Support Tool Module is designed to provide a manageable list of potential deficiencies/conditions for the user to consider (e.g., one hundred or less, fifty or less, fifteen or less, one, etc., or any number or range therebetween) and it remains the user's responsibility to establish the diagnosis. In this manner, the Diagnostic Support Tool Module supports the user in identifying and establishing the diagnosis, but ultimately leaves it up to the user to practice medicine.

A user 202 (typically a healthcare provider user 202a) may access the Diagnosis Support Tool Module of the system 10 by clicking on Diagnosis Support Tool tab 114 of the homepage interface 100 (or any other interface described herein comprising tab 114 or a similar link to access the functionality of the Diagnosis Support Tool Module). As previously described in the general overview of the system 10, the Diagnosis Support Tool Module is delivered, at least in part, by the processor(s) 20 executing one or more applications 18 of the system 10, where each of the application(s) 18 are written to achieve the goals of the present disclosure.

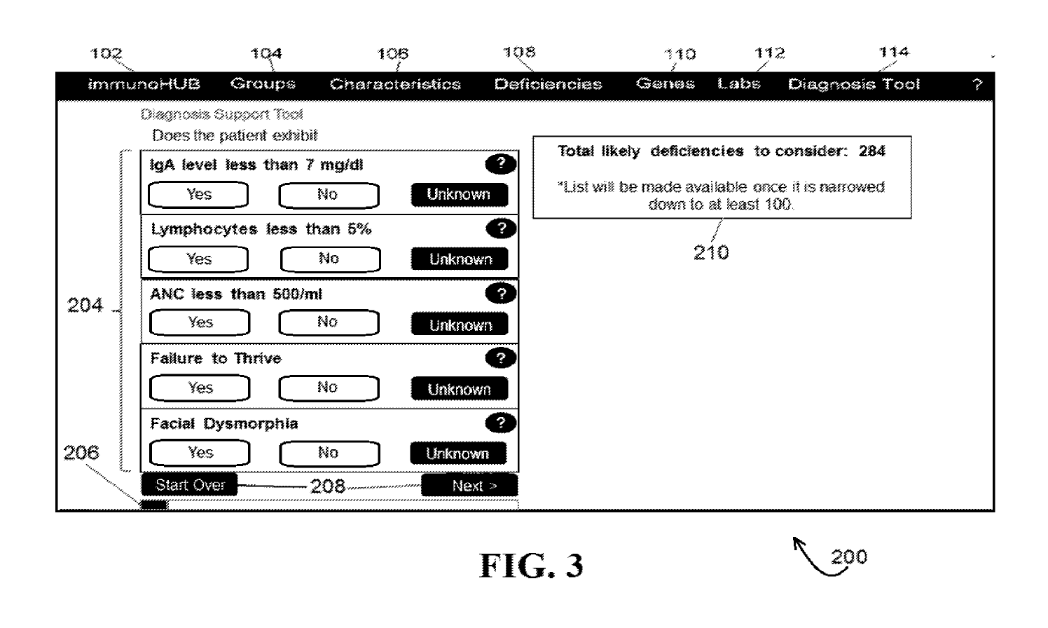

FIG. 3 depicts at least one example of a user interface associated with the Diagnosis Support Tool Module—an inquiry page 200. Here, inquiry page 200 comprises a list of inquiries 204, progress bar 206, one or more navigation buttons 208, and potential medical conditions information display 210. In its simplest form, however, when a user 202 accesses the Diagnosis Support Tool Module, the Diagnosis Support Tool Module simply requests information from the user 202 regarding the patient at issue. Such request may take the form of the initial list of inquiries 204 regarding the patient at issue as shown in FIG. 3 in connection with inquiry page 200, or may comprise any other interface or input request now known or hereinafter developed capable of achieving such purpose.

In general, the list of inquiries 204 comprises a list of diagnostic-centric questions regarding the patient at issue. For example, in at least one embodiment, the list of inquiries 204 requests “key indicator data” (e.g., physical examination findings, laboratory results, chromosome analysis data, etc.), pathognomonic data (i.e. inquiries regarding whether or not the subject exhibits certain characteristics that may be specific to a particular disease condition), related environmental data, and/or any other data or input related to the patient at issue and/or that may be useful to distinguish between indicators and/or symptoms of two or more medical conditions. In at least one exemplary embodiment, the list of inquiries 204 used by the Diagnosis Support Tool Module is not presented to the user 202 all at once, but rather divided up into sets of inquiries presented in succession. The initial list of inquiries 204 displayed to a user 202 may comprise a standard list that is always initially displayed and/or the entire list of inquiries 204 may comprise a predetermined, static list. Alternatively, in at least one embodiment and as described in further detail herein, the list of inquiries 204 may change following the initial list of inquiries 204, and/or be customized, depending on a user's 202 previously submitted response(s). This updatable or dynamic embodiment may be achieved by an administrator user 202b or the system 10 itself—via an application 18, for example.

As shown in FIG. 3, a user 202 may submit a response to a list of inquiries 204 using buttons (e.g., “Yes,” “No,” and “Unknown” or “Maybe”); however, any other interface element input controls (such as, for example, checkboxes, radio buttons, dropdown lists, toggles, text fields, date fields, etc.) or combinations thereof may be employed. In at least one alternative embodiment, options for response to an inquiry may be a field to enter free-form text, a field to enter/select provided text, buttons to select a value or applicable range, an option to upload diagnostic test results, images, and the like, etc. Indeed, the option for response associated with any inquiry may be customized as desired to enable a user to input the data requested into the system 10. Inquiry page 200 may also include one or more links to additional information associated with each inquiry of the list of inquiries 204. For example, a user 202 may select an additional information link associated with a particular inquiry, which will provide the user 202 with explanatory detail as to the inquiry itself (e.g., specify each “key indicator” question) either through a separate interface, in a pop-up, or using other mechanisms known in the art.

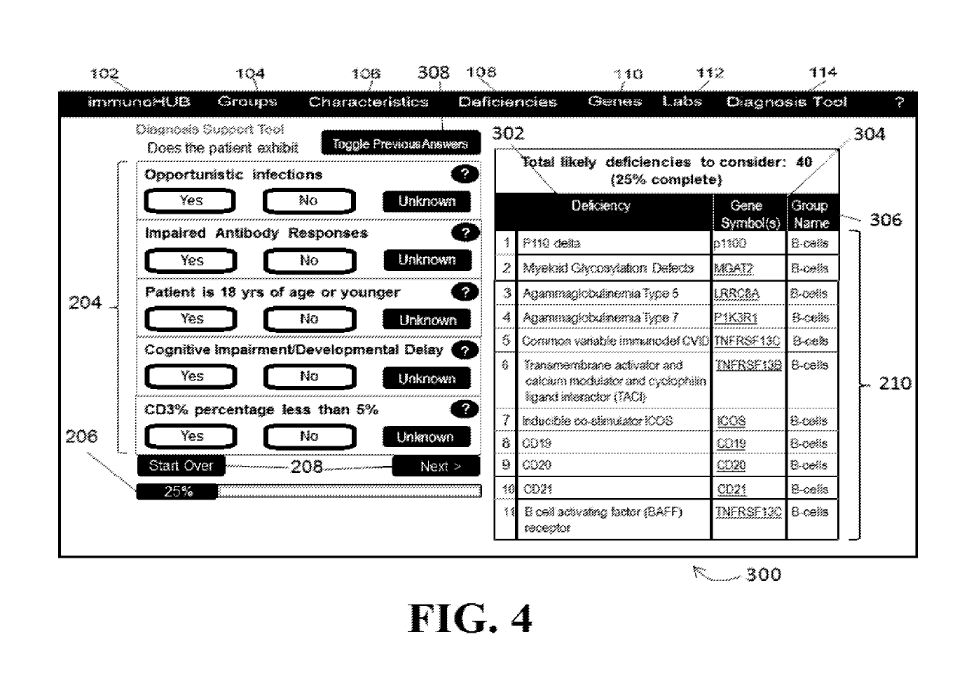

The inquiry page 200 may also comprise a progress bar 206 indicative of a user's 202 progress through the diagnostic process as supported by the Diagnosis Support Tool Module. Navigation button(s) 208 may also be displayed to facilitate a user's 202 navigation through the Module and his or her access to the functionality thereof. Additionally, the inquiry page 200 may also comprise a potential medical conditions-information display 210. To facilitate ease of use and understanding on the part of the user 202, in at least one exemplary embodiment, if the number of conditions that correlate with the input data (or lack thereof) are deemed to be too high in number to be clearly displayed on the inquiry page 200, the information display 210 lists the number of potential conditions (or a range thereof) that currently match the inquiry. The inquiry page 200 of FIG. 3 illustrates this feature. There, because the inquiry page 200 is the initial inquiry page 200, the Module has not yet eliminated or selected any potential medical conditions from the reference database 13 of the system 10. As such, instead of listing all of the medical conditions stored in the reference database 13, the information display 210 identifies that there is a total of 284 deficiencies to consider. Once the Module begins to narrow this list down based on the user's 202 input data and the algorithms of the Module, the information display 210 will then provide a list of specific medical conditions that are potentials for diagnosis (see, for example, the information display 210 of FIG. 4).

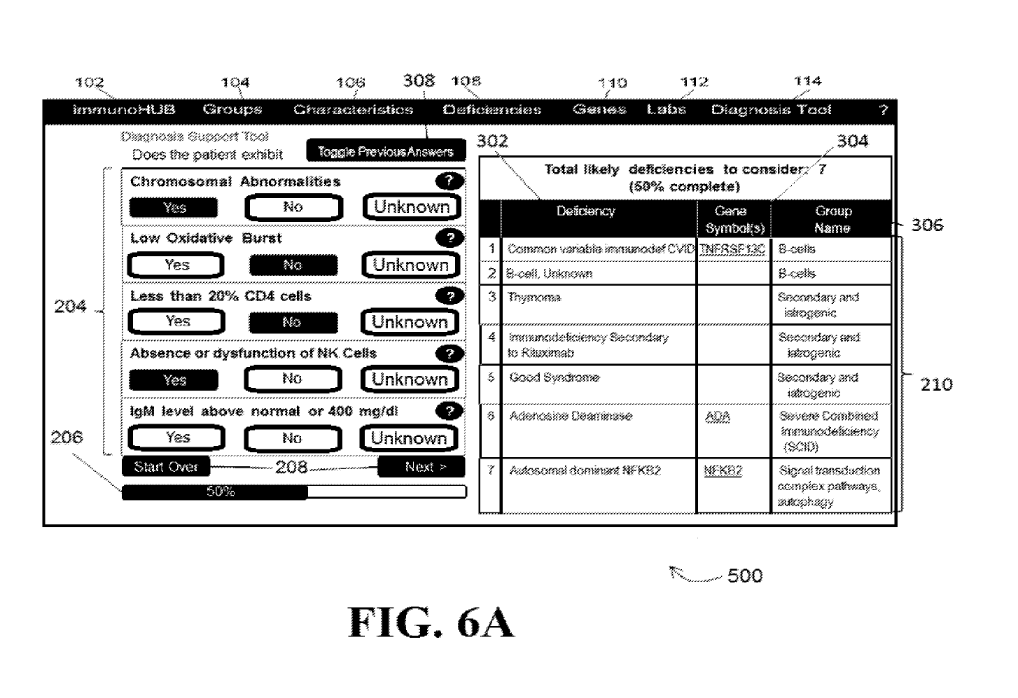

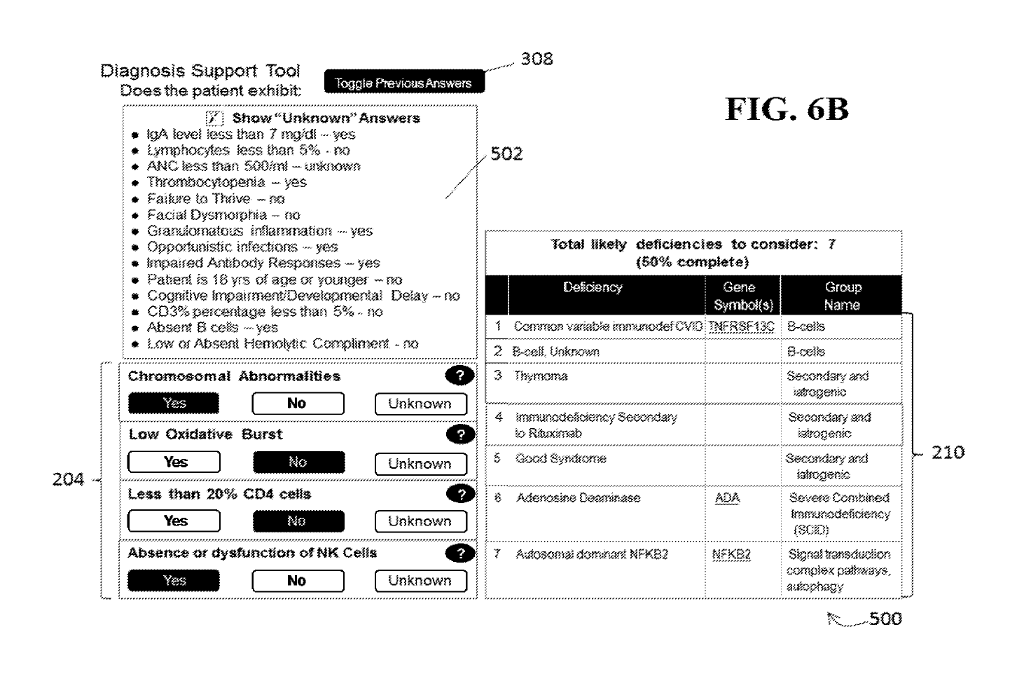

Now referring to FIGS. 4, 6A, and 6B, embodiments of additional user interfaces of the Diagnosis Support Tool Module are shown, where the user 202 has previously submitted input data and the analysis process has run for at least one cycle. For example, inquiry page 300 of FIG. 4 and inquiry page 500 of FIG. 6A both display updated inquiry lists 204 (each comprising a new list of inquiries) and updated subsets of medical conditions within the information display 210 that the Module formulated based on the user's 202 previously submitted responses. By way of comparison, inquiry page 300 represents an example of an inquiry page associated with a user 200 who is 25% through the diagnostic process using the Diagnosis Support Tool Module, whereas inquiry page 500 represents an example of an inquiry page indicative of a user 202 who is 50% (or half-way) through the diagnostic process using the Diagnosis Support Tool Module. Notably, the questions within the updated inquiry lists 204 are different (i.e. “updated”) and the number of deficiencies that are under consideration (i.e. included within the subset) decrease as the diagnostic process progresses (40 conditions within the subset for inquiry page 300 as compared to 7 for inquiry page 500). Furthermore, updated inquiry pages 300, 500 may additionally comprise button 308, the selection/activation of which causes the user's 202 previously submitted inquiry responses to be displayed for reference (see window 502 of FIG. 6B).

As previously noted, when the identified subset of potential medical conditions comprises a number of conditions that is less than a predetermined amount (here, for example, 100), a list of the particular medical conditions included within the identified subset may be provided to the user 202 in information display 210. FIG. 4, for example, shows that the identified subset includes 40 medical conditions, with each of those conditions specifically listed. In such embodiments, information display 210 may include the name of the deficiency 302, the gene(s) associated with the deficiency 304 (if any), the group name associated with each deficiency 306, and any other information that may be desired. Furthermore, if a user 202 desires additional information on one or more of the genes 306, each gene 306 listed can include a link to additional detail specific thereto (accessible, for example, by clicking a link present within the column).

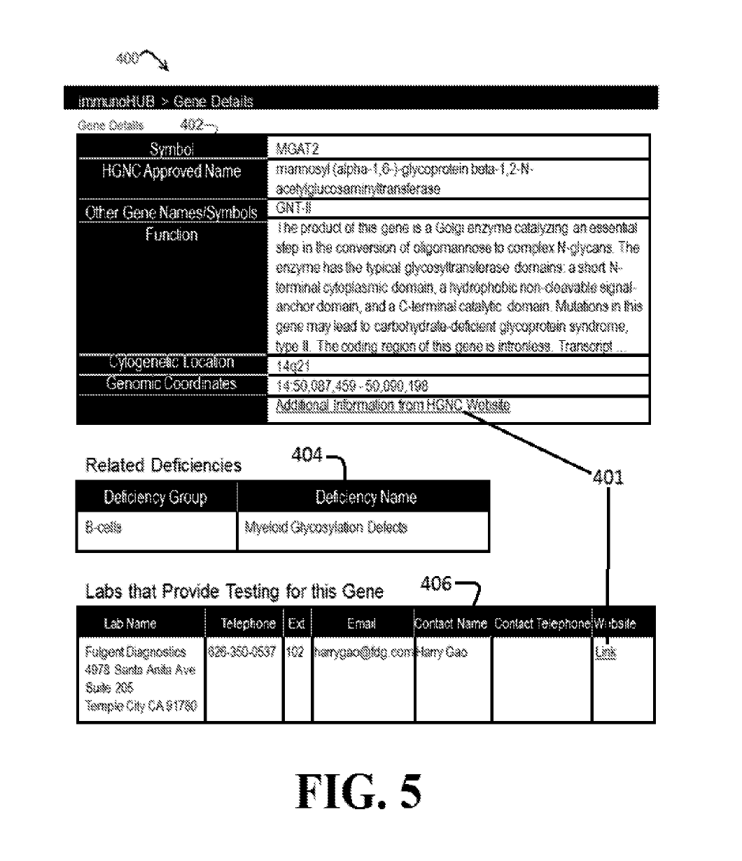

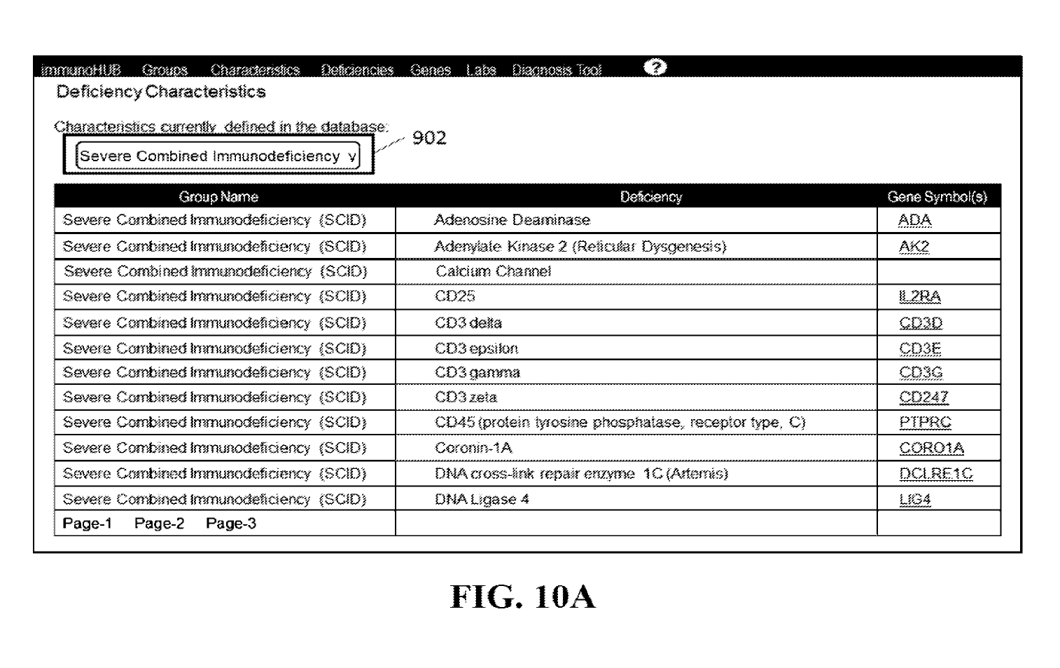

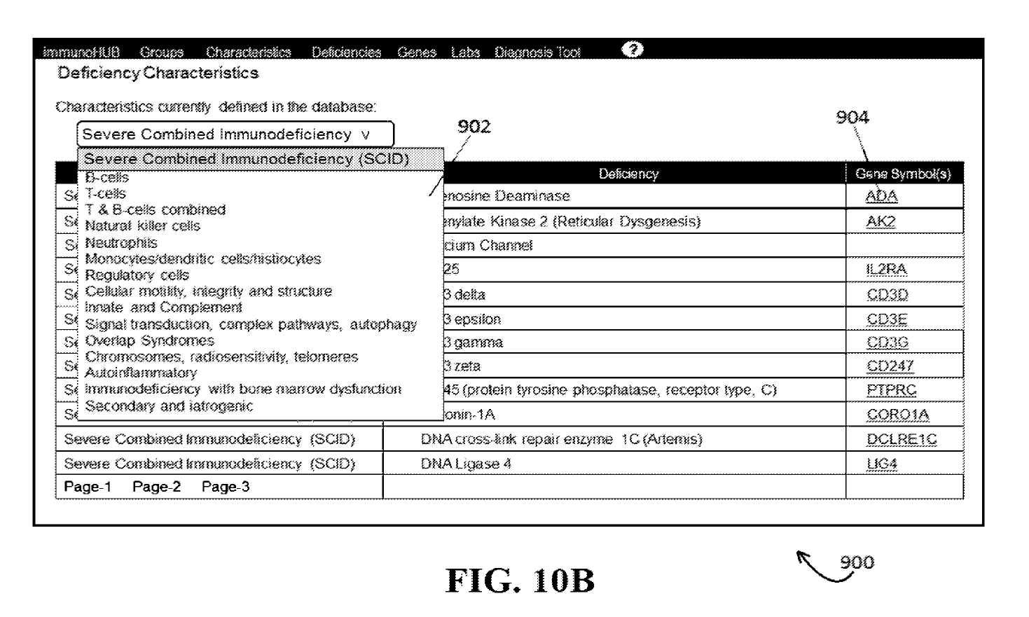

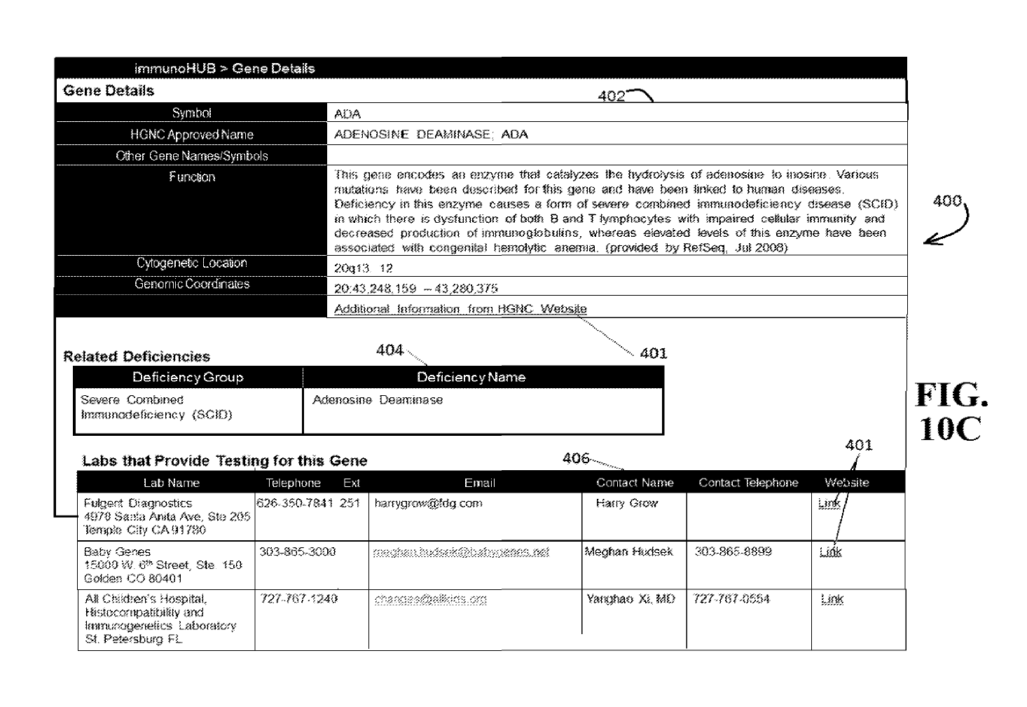

FIG. 5 shows a representative example of a gene detail page 400 for gene MGAT2 that may be accessed through the Diagnosis Support Tool Module by clicking the MGAT2 link displayed in column 304 of information display 210 of FIG. 4 (see also FIG. 10C). In that at least one exemplary embodiment shown in FIG. 5, the gene detail page 400 may include information regarding the gene itself (402), a list of deficiencies related to the gene (404), and a list of laboratories that provide testing for the gene (406); however, any detail related to a particular gene may be included as desired. Furthermore, the gene detail page 400 may also include links 401 to third party websites and/or information related to the particular gene of interest.

The gene information displayed in the gene detail page 400 may be stored in a database or other memory of or accessible by a server 12 of the system 10 (including, without limitation, a third-party database). Where the gene information is stored within the system 10, such information may be updated by an administrator user 202b (for example, one having the appropriate credentials) such that the information can be kept current to ensure the accuracy thereof. Notably, in at least one exemplary embodiment described in further detail herein, the gene detail pages 400 originates from a Genes Module of the system 10 and, as such, the Genes Module and Diagnosis Support Tool Module interface such that the gene detail page 400 is accessible by a user 202 directly through the Diagnosis Support Tool Module.

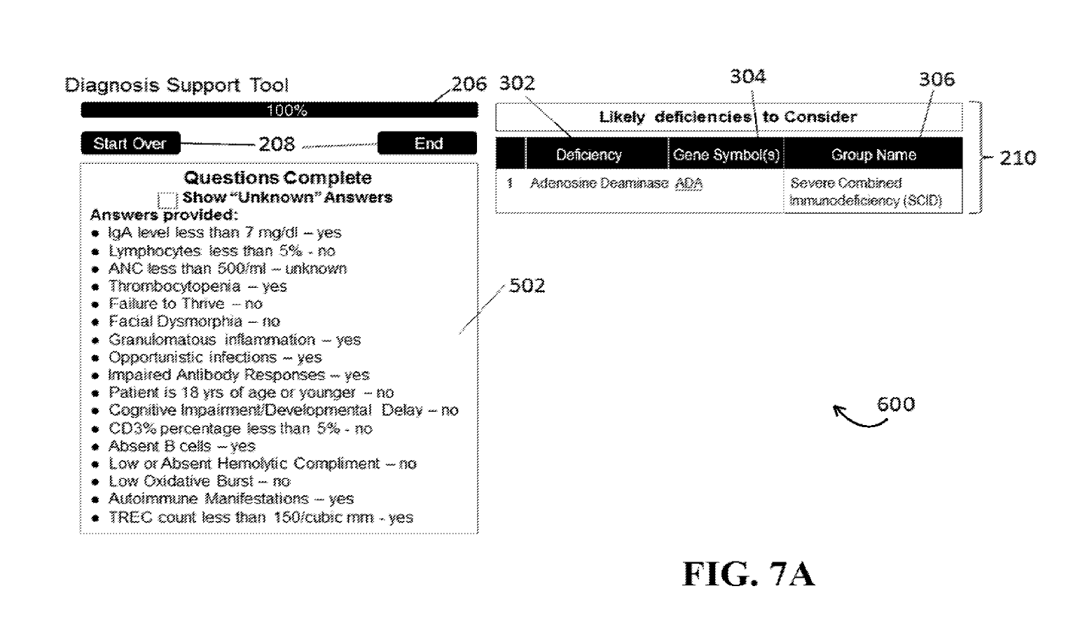

Now referring to FIG. 7A, an example of yet another user interface associated with the Diagnosis Support Tool Module is shown—diagnosis page 600. The Module displays the diagnosis page 600 to a user 202 when the diagnosis support process is complete (i.e. either the subset of medical conditions cannot be narrowed any further or an additional list of inquiries 204 cannot be generated). As shown in FIG. 7A, a single medical condition is listed in information display 210, which comprises a potential diagnosis (i.e. this condition properly correlates with all input data submitted by the user 202).

Referring now to the background process that occurs when the Diagnosis Support Tool Module is used, the steps hereof will now be described as method 750 (see FIG. 7B) using the interfaces 200-600 by way of explanatory examples. Accordingly, when a user 202 accesses the Diagnosis Support Tool Module at step 752, initial inquiry page 200 may be shown, in which an initial list of inquiries 204 is presented. After the user 202 inputs and submits data in response to the list of inquiries 204 at step 754 (either by hitting the “Next” button 208 or through other means now known in the art or hereinafter developed), a processor 20 of the server 12—and/or, in certain embodiments, a processor 20 of the client 14—executes one or more applications 18 at step 756 that access and compare the data received from the user (the “input data”) with the medical reference database 13 pursuant to at least one algorithm. Perhaps more specifically, at step 756 the input data is compared against the details of each medical condition within the medical reference database 13 and a subset of medical conditions that properly correlate with the input data is identified at step 758.

In at least one exemplary embodiment, the initial list of inquiries 204 presented at step 752 (seven (7) inquiries displayed on inquiry page 200, for example) comprises questions related to patient key indicators such as the patient's past medical history, the patient's current condition assessed via physical examination or screening, laboratory results, etc., and the algorithm is a negative selection algorithm. There, execution of the application(s) 18 at step 756 maps and/or analyzes (e.g., through categorizing, sorting, etc.) the data within the medical reference database 13, correlates the input data with the mapped reference data, and rejects or discounts any potential medical conditions in the reference database 13 that are not associated with the key indicator(s) or pathognomonic data indicated by the input data (e.g., conditions with a definitive “no” response for a key indicator). This identifies a resulting subset of medical conditions at step 758 that are associated with the input data (e.g., conditions with a definitive “yes” response for one or more key indicators). In at least one embodiment, if there is a potential for a key indicator to be positive, but not definitive of a potential condition, the condition is marked as “maybe” or “unknown” by the application 18 and remains in the resulting subset of medical conditions. Furthermore, the system takes this unknown or maybe response into account when narrowing down the subset of potential medical conditions and, in at least one embodiment, in formulating the next round of inquiries.

It will be appreciated that any algorithm or method of comparison may be employed, provided a subset of medical conditions is generated that accurately correlates with the input data. The subset of medical conditions identified by the Diagnosis Support Tool Module is then displayed at step 760 to the user 202 within the information display 210 and an updated list of inquiries 204 is provided to the user 202 (see FIGS. 4, 6A, and 6B). Additionally, in those embodiments of the Diagnosis Support Tool Module that includes dynamic lists of inquiries 204, at step 762 an application 18 executed by the processor 20 evaluates the most recent grouping or subset of medical conditions and the remaining unknowns with respect to the current subject to formulate an updated list of inquiries 204 to be presented to the user 202 at step 764.

If the application 18 deems that an inquiry will not be useful to further narrow the number of potential conditions within the subset—either in light of the previously input data and/or the remaining medical conditions in the previous subset—such inquiry is not presented to the user 202 and the then-current subset of medical conditions is finalized at step 763. In this manner, the Diagnosis Support Tool Module promotes efficiency, directs the diagnostic focus, and prevents the user 202 (and the patient) from being inundated with ancillary tests and labs that are not necessary or helpful.

The user 202 can review the medical conditions listed in the identified subset of potential medical conditions (see information display 210 of FIG. 4) and/or provide additional input to further narrow the identified subset of medical conditions by responding to the updated list of inquiries 204 at step 766. Accordingly, the process continues with: (a) the user 202 submitting additional input data in response to the updated list of inquiries 204 at step 766, (b) the Module comparing the new input data against the details of each medical condition within the previously identified subset of medical conditions pursuant to a defined algorithm at step 762; and (c) the Module identifying a new, updated subset of medical conditions and generating and/or displaying an updated list of inquiries 204 to the user 202 at step 764. The Module then repeats steps 762-766 until at least a manageable group of likely medical conditions associated with the input data submitted by the user 202 is achieved and the then-current subset of medical conditions is finalized at step 763.

In at least one exemplary embodiment, a subsequent list of inquiries 204 presented to the user 202 at step 760 comprises inquiries related to pathognomonic data (i.e. includes questions regarding the patient's current condition) and subsequent comparison step 762 performed by the Module utilizes a positive selection algorithm such that only those conditions within the identified subset that are associated with the new input data are selected for the updated identified subset of potential medical conditions. This is particularly useful once the subset of potential medical conditions has been narrowed to a manageable group as pathognomonic data, in particular, is very specific to certain medical conditions and often times can point a healthcare provider to a diagnosis (when taken in conjunction with the previously performed iterations of the method 750 steps).

However, it will also be appreciated that the subsequent list(s) of inquiries 204 (or portions thereof) may be generated at step 764 based on an analysis of the medical conditions present within the most-recent identified subset of medical conditions. As previously stated, in at least one exemplary embodiment, step 762 may comprise the processor 20 executing an application 18 that employs a data-driven logic (a dynamic data driven application, for example) to analyze and/or map the medical conditions/characteristics of the most-recently identified subset of medical conditions, identify any patterns, potential patterns, or the lack of patterns of disease characteristics or key indicators therein or in the input data, and generate a list of inquiries 204 designed to request additional data from the user 202 that will most efficiently distinguish between the medical conditions of the identified subset and/or confirm or eliminate a potential diagnosis. In this manner, key indicators and data requests that will not further narrow the list are not asked and the Diagnosis Support Tool Module can personalize the evaluation to the patient at issue and target further analysis to the most relevant immune cell or pathway by way of generating focused lists of inquiries 204 and/or testing recommendations.

Along these same lines, not only can the Diagnostic Support Tool Module dynamically generate lists of inquiries 204 in response to user 202 input data (step 762), but it may also indicate (and/or suggest) to the user 202 additional diagnostic tests to run at optional step 765a, the results of which may be entered at step 766, be beneficial in efficiently moving towards a manageable and comparatively narrow subset of potential medical conditions (e.g., fifteen or less) and, ultimately, may assist the user 202 to establish an efficient and accurate diagnosis. Furthermore, in at least one exemplary embodiment, at optional step 765b, the Diagnostic Support Tool Module interfaces with the Laboratory Module of the system 10 (described in additional detail below) to provide the user 202 with information on particular laboratories that perform the identified tests and even potentially schedule the tests online where a selected third-party laboratory is in communication with the system 10 over the network 16.

One or more of the applications 18 that allow for the automatic and dynamic generation of lists of inquiries at step 762 and/or test suggestions at optional step 765a may, in at least one exemplary embodiment, comprise a machine-learning service. Such a machine-learning service may utilize machine-learning statistical analysis to provide additional insights regarding the usefulness and incisiveness of each key indicator and/or the data within the medical reference database 13. Where employed, the machine-learning service can communicate with the other applications 18 and the medical reference database 13 via an interface (e.g., an Application Program Interface (API)) or as is otherwise known in the art, with such interface providing access to one or more commonly-used machine adaption techniques. For example, an API can provide access to interfaces for ranking, clustering, classifying, and prediction techniques such as autonomous pattern recognition, decision tree learning, inductive logic programming, similarity metric learning, clustering, Bayesian network analysis, and/or the like. Additionally, or alternatively, the system 10 can be configured such that a user 202 providing input data into the system 10 (through the Diagnosis Support Tool Module or simply by updating the medical reference database 13) provides input to the machine-learning service.

Still further, the machine-learning service can also include a data aggregation and representation engine or the like that consistently receives and stores input data, perhaps from multiple sources and/or as part of the medical reference database 13. The stored input data can be aggregated to discover features within the data, such as correlations between phenotypes, function or genetic pathways, and certain disorders. In certain embodiments, the machine-learning service utilizes network support functionality to access data aggregated across multiple platforms. For example, the machine-learning service may interface with the medical reference database 13 as well as databases external to the system 10 (e.g., third party databases and/or public databases). Such aggregated data can be stored in one or more of the servers 12, or on the clients 14, and accessed as needed. For example, the aggregated data can be used to train and/or set initial values for the machine adaptation techniques used by the machine-learning service at step 762 as part of generating inquiry lists, at step 765a in connection with generating testing recommendations and/or in steps 756 and 762 in analyzing the data within the medical reference database 13 in light of user 202 input.

In addition to the Diagnosis Support Tool Module, the education and diagnostic support system 10 may also comprise a variety of other Modules geared towards education and providing explanatory data/information to a user 202. Each of these Modules may be interfaced with the Diagnosis Support Tool Module such that relevant information therein can be accessed directly from the Diagnosis Support Tool Module by a user 202 as well as via homepage interface 100.

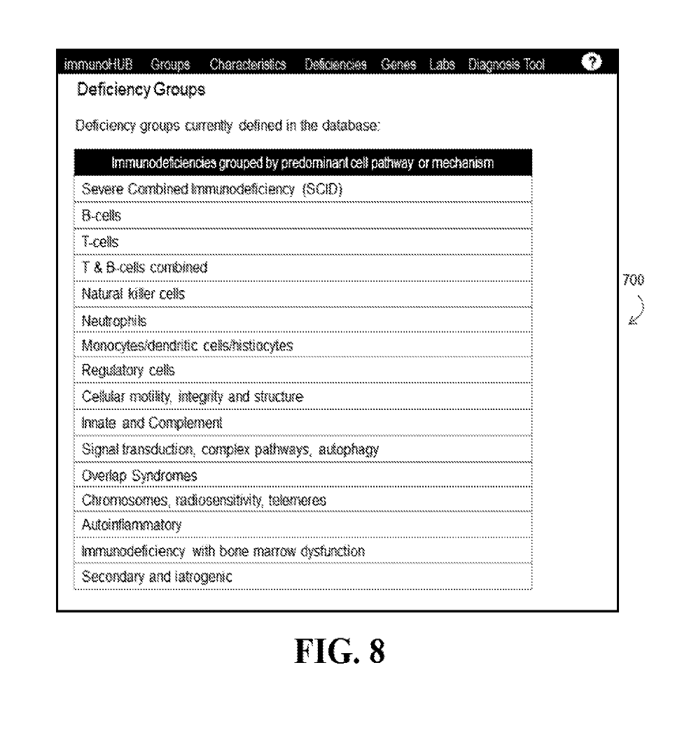

An example of a user interface associated with a Groups Module is shown in FIG. 8—deficiency groups page 700. In effect, the Groups Module facilitates education and understanding of the targeted deficiencies or disorders. The Groups Module provides a novel and straight-forward classification of targeted deficiencies or disorders by, for example, the predominant component that is altered by the presence of an associated molecular abnormality or any other classification criteria. This is significant because many disorders and/or diseases can be summarized as the coordinated upregulation and downregulation of a particular gene or via other factors.

For example, immunological function can be summarized as the coordinated upregulation and downregulation of a body's host defense against disease-causing organisms. This host defense system is complex and can comprise a vast variety of tissues and cellular components including specialized cells (e.g., T-cells and subsets thereof), organelles, transcription factors, proteins (i.e. antibodies), growth factors including cytokines, transmembrane-to-nucleus signaling pathways, and cell movement and trafficking apparatus. Indeed, while many primary immunodeficiencies affect more than one cell type, pathway, or mechanism of regulation, conventional practice has been to categorize such conditions based on the predominate cell type or function that is compromised (such as T cell, B cell, T and B cell deficiencies, phagocytic disorders, etc.). Here, the Groups Module can be used to provide clarity to these deficiencies and facilitate a user's understanding of the same.

A Characteristics Module is also provided (accessible through Characteristics tab 106). Individuals with similar deficiencies, diseases, or disorders are likely to exhibit patterns of characteristics that are different from individuals without such deficiencies diseases, or disorders. As such, the likelihood of a deficiency, disease, or disorder in an individual can be estimated by the presence or absence of a combination of characteristics. A representative interface 800 associated with the Characteristics Module comprises a list of characteristic findings that are potentially indicative of primary immunodeficiencies when identified as part of a patient's medical history, physical examination and laboratory evaluation.

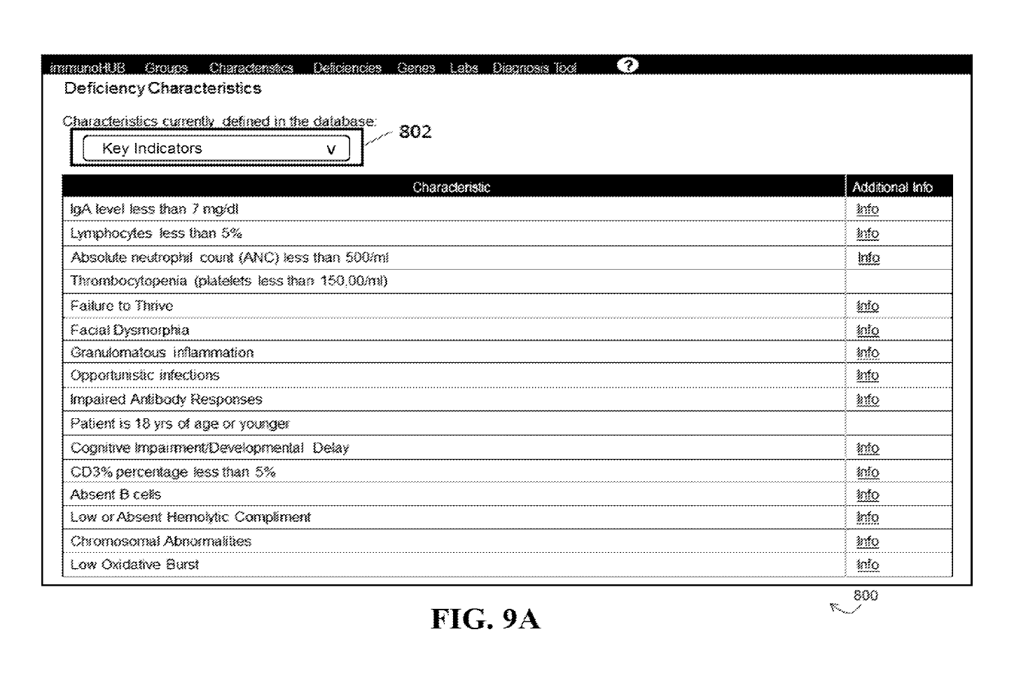

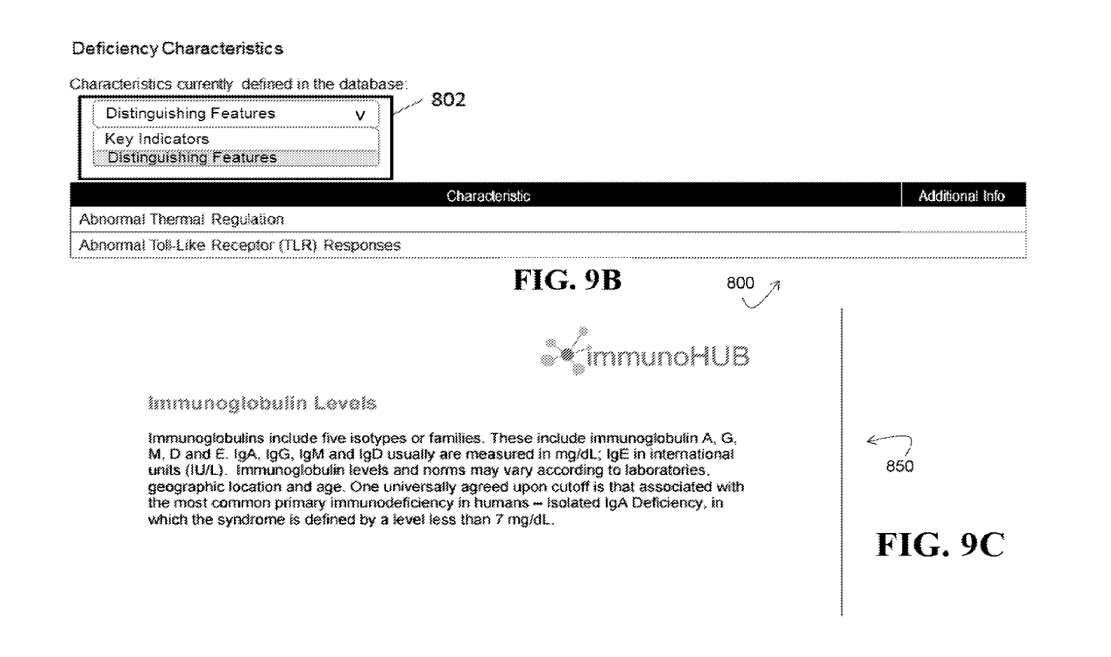

As shown in FIGS. 9A and 9B, such deficiency characteristics can be divided between key indicators and distinguishing features (see dropdown list 802). Additionally, the Characteristics Module can provide links to additional detail regarding particular “key indicators” or distinguishing features (see page 850 of FIG. 9C). It will be appreciated that such additional detail may be stored within a database of the system 10 itself, in storage that is accessible thereby, and/or simply be included within a third-party database to which the system 10 is linked via the network 16.

A Deficiencies Module (accessible through Deficiencies tab 108 or otherwise) may also be provided. User interface 900, shown in FIGS. 10A and 10B, may be used in connection therewith. The Deficiencies Module of the system 10 comprises a database of all diseases of note with respect to the system 10, as well as their associated conditions, phenotypic manifestations, characteristics, any defined molecular causes, and categories (see dropdown menu 902). The Deficiencies Module is easily updatable, which allows for the integration of recently described conditions and to maintain currency. For example, clinical expertise and information from physicians, other healthcare providers, and scientific advisers can be easily added to the underlying databases of or accessible to the system 10 (e.g., the medical reference database 13 or other databases of the system 10) via the Internet or otherwise. Such databases may also be populated from (or by linking to) literature, scientific journals, text books, encyclopedias, scientific community list-servs, posters, abstracts, presentations, and patient case reports of patients affected by a certain group of diseases or deficiencies. In at least one embodiment, individual deficiencies or diseases are identified, and the associated names, molecular definitions (genes causing them), and other information are provided. Additionally, each deficiency and/or disease may be categorized within the database in a manner that facilitates its usefulness in connection with the various functionalities of the system 10—for example, a primary immunodeficiency may be categorized by immunity function pathway or genetic pathway. Classification and/or categorization may be assigned upon the initial upload of information to the system 10 or by one or more applications 13 during operation of the herein described processes.

In at least one exemplary embodiment, the Deficiencies Module either interacts with and is populated from the medical reference database 13 of the system 10 or is in communication therewith such that it is critical to the operation of the Diagnosis Support Tool Module as described herein. Additionally, the Deficiencies Module may be accessed and used by a user 202 directly as an education resource (via, for example, Deficiencies tab 108). It will be appreciated that the Deficiencies Module may be interfaced and/or linked with the Genes Module such that links 904 of the deficiency module user interface 900 navigates to a gene detail page 400 of the Genes Module as shown in FIG. 10C.

To date, there are more than 285 primary immunodeficiency diseases that have been described in the medical literature, most of which have defined molecular causes and are categorized by disease group. Some of the immunodeficiency diseases are caused by different molecular genotypes that have similar human phenotypes and, as such, individuals with similar genotypes will display similar (but not identical) phenotypes. Thus, although there is a range of characteristics that overlap from one particular immunodeficiency disease to another, subjects that display a certain combination of characteristics segregate a likely diagnosis from a less likely diagnosis, which further facilitates an efficient molecular diagnostic focus.

Due, at least in part, to the analytical and comparative processes utilized by the Diagnosis Support Tool Module and the easily updatable configuration of the Deficiency Module, the education and diagnostic support system 10 described herein is particularly well-suited for use with immunodeficiency diseases. Indeed, the Deficiency Module may include all of the currently-identified immunodeficiency diseases and their associated characteristics (including molecular genotypes and human phenotypes), which are then taken into account during operation of the Diagnosis Support Tool Module. As such, the education and diagnostic support system 10 hereof can personalize an evaluation and target further analysis to the most relevant immune cell or pathway. Notwithstanding the foregoing, the education and diagnostic support system 10 can also be directed towards other medical areas such as infectious diseases, hematology, oncology, rheumatology, and any other medical specialty or field, simply by focusing the data within the associated database(s) on such areas. The system hereof may additionally or alternatively include medical sub-specialties, such as immunohematology, immune-oncology, and the like. However, the education and diagnostic support system 10 need not be limited to any one medical specialty or field, but instead may span as many medical disorders, deficiencies, and/or diseases as key indicators and other data may be saved into the medical reference database 13 or accessed by the system 10. Indeed, the breadth in application of any particular system or method of the present disclosure is only limited by user preference, the size of the database(s) available, and the medical information available.

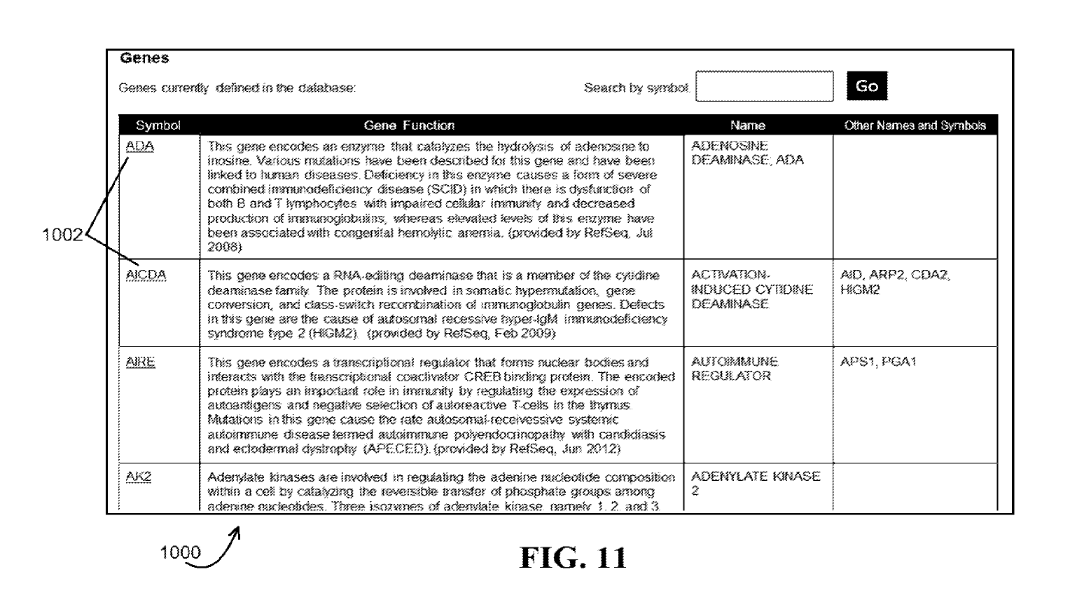

As previously stated, the education and diagnostic support system 10 further comprises a Genes Module (accessible from various components of the system 10 including, without limitation, via Genes tab 110 from the homepage interface 100, the Diagnosis Support Tool Module, and/or through the Deficiencies Module). It is well established that certain genes affect the function of the human immune system as demonstrated by an increased susceptibility of those individuals with mutations to experience recurrent and/or severe infections, opportunistic infections, autoimmune disease, autoinflammatory illness, and/or cancer. As such, the Gene Module of the system 10 provides a database of gene information that is available to users 202 for educational and other purposes. Gene module interface 1000 illustrated in FIG. 11 shows at least one embodiment of an interface of the Gene Module that displays a list of relevant genes, their associated function, name, symbols, and other information. Accessing link 1002 for a particular gene navigates a user to the gene detail page 400 for such gene, which provides further detail regarding the gene of interest (see FIGS. 5 and 10C).

Additionally, since many genes and their relationship(s) to human illness have been recently discovered, a single, predominant nomenclature for the same has not yet been established, especially as they apply to the field of immunology. As such, in addition to providing information to users 202 regarding relevant genes, the Genes Modules of the system 10 are designated to match the nomenclature adopted by the Hugo Gene Nomenclature Committee (HGNC) for consistency purposes, and links to HGNC's third party website are provided.

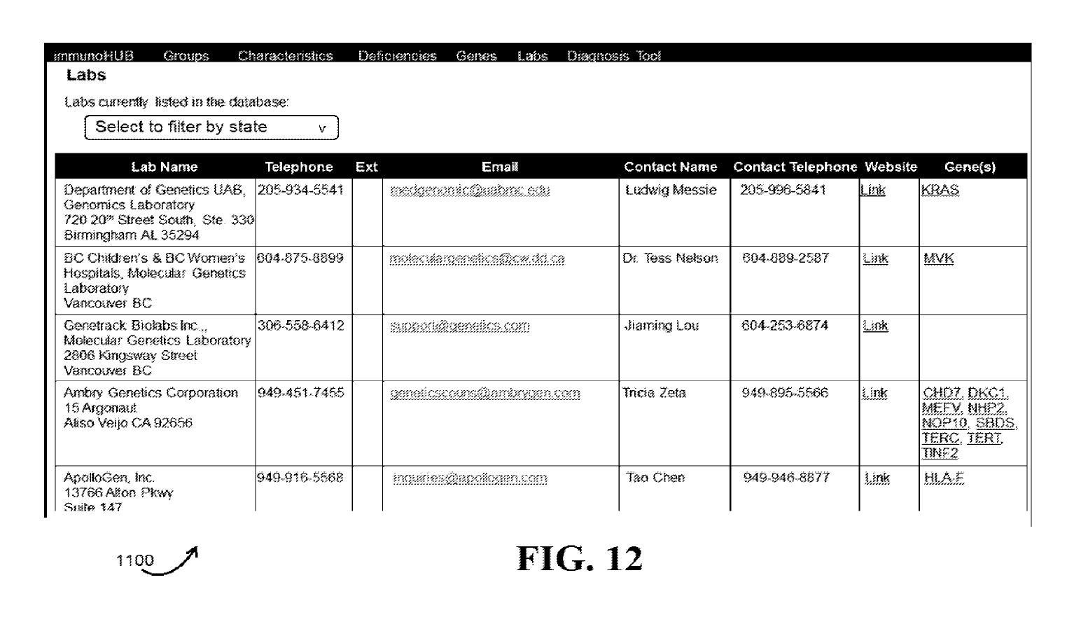

A Laboratories Module is also provided in the education and diagnostic support system 10 to provide a user 202 with rapid access to one or more facilities that are certified to perform particular types of analyses. Perhaps more specifically, the Laboratories Module comprises a list of laboratories that perform analyses and tests relevant to an analysis performed by the Diagnosis Support Tool Module (see interface 1100 of FIG. 12) that may be sorted pursuant to desired filters or categories (e.g., location). In at least one exemplary embodiment, the list of laboratories comprises those that provide mutational analyses and gene sequencing. The names and contact information of such laboratories may be included to facilitate user 202 contact to either confirm that the desired test(s) is/are available, or to seek guidance regarding details of sample collection, packaging, and delivery. Furthermore, in at least one exemplary embodiment of the system 10, a third-party website or system associated with a particular laboratory may be accessible through and/or interfaced with the system 10 via the network 16. In this manner, a user 202 can select a laboratory from the Laboratory Module and access their website or system to communicate therewith, schedule an analysis, etc. without logging off or leaving the education and diagnostic support system 10.

As previously described, the Laboratories Module may be interfaced with the Diagnosis Support Tool Module and/or accessed via Laboratories tab 112. Furthermore, the list of laboratories is customizable such that any not listed can be added upon request.

While various embodiments of the systems for education and diagnosis, and methods of using the same have been described in considerable detail herein, the embodiments are merely offered as non-limiting examples of the disclosure. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method for treating a medical condition detected in a subject, the method comprising the steps of:
(a) displaying a list of inquiries to a user, the list of inquiries formulated to distinguish between key indicators of a plurality of medical conditions as compared to a healthy subject;
(b) receiving, on a server, a set of data from a user, the set of data regarding a subject and in response to the list of inquiries;
(c) executing a first application by a processor to reference the set of data received against a reference database and identify a subset of medical conditions pursuant to a first algorithm, the reference database comprising a plurality of medical conditions and associated key indicators and data associated with each medical condition, and the identified subset of medical conditions comprising medical conditions that correlate with the received set of data;
(d) executing at least a second application by the processor to:
generate an updated list of inquiries to distinguish between the medical conditions of the identified subset, and
transmit the updated list to the user over the network;
(e) receiving, on the server, a subsequent set of data from the user, the subsequent set of data in response to the updated list of inquiries;
(f) repeating steps (c)-(e) unless and until the identified subset of medical conditions either consists of a manageable group of medical conditions or an updated list of inquiries cannot be generated due to lack of distinction between the key indicators and data of each medical condition of the identified subset;
(g) referencing the subsequent set of data against the identified subset of medical conditions and, pursuant to a second algorithm executed by the processor, identifying a second subset of medical conditions therein that correlate with the subsequent set of data received from the user; and
(f) treating the subject for a diagnosed medical condition selected from the identified second subset of medical conditions.

2.-3. (canceled)

4. The method of claim 1, wherein the manageable group of medical conditions comprises fifteen or less medical conditions.

5. (canceled)

6. The method of claim 1, wherein the medical conditions are selected from a group consisting of conditions characterized by deficiency of immune function or regulation, autoimmune diseases, auto-inflammatory diseases, and infectious diseases.

7. The method of claim 6, wherein:
the conditions characterized by deficiency of immune function comprise primary immunodeficiency conditions or non-primary immune-mediated conditions, the auto-inflammatory diseases comprise rheumatologic conditions, or both the conditions characterized by deficiency of immune function comprise primary immunodeficiency conditions or non-primary immune-mediated conditions, the auto-inflammatory diseases comprise rheumatologic conditions; and
the medical conditions comprise general medicine and pediatric conditions.

8.-9. (canceled)

10. The method of claim 1, wherein the first algorithm is a negative selection algorithm such that the step of executing a first application by a processor to reference the set of data against a reference database further comprises disregarding those medical conditions that do not correlate with the set of data.

11. (canceled)

12. The method of claim 1, wherein the data set of data comprises key indicator data comprising physical examination findings, laboratory results, and/or chromosomal analysis data.

13. The method of claim 1, wherein the second algorithm is a positive selection algorithm and the subsequent set of data received comprises pathognomonic data exhibited by the subject.

14. (canceled)

15. The method of claim 1, wherein step (d) further comprises generating the updated list of inquiries based on distinctions identified by a third application between the key indicators and data associated with each medical condition of the identified subset.

16. (canceled)

17. The method of claim 15, wherein generating the updated list of inquiries is performed automatically by a third application comprising a machine-learning service, wherein the machine-learning service analyzes the reference database comprising the plurality of medical conditions and their associated key indicators and data using a statistical analysis methodology selected from a group consisting of decision tree learning, inductive logic programming, similarity metric learning, clustering, and Bayesian network analysis.

18. The method of claim 1, further comprising the step of executing a fourth application by the processor to recommend one or more diagnostic tests, the results of which will be useful in distinguishing between the medical conditions of the identified subset.

19. The method of claim 1, further comprising the step of performing a diagnostic test on the subject, wherein the subsequent set of data comprises results of the diagnostic test.

20. The method of claim 1, further comprising the steps of:
receiving, on the server, a request from the user to schedule a diagnostic test with a laboratory;
executing an application by the processor to submit a request, over the network, to the laboratory to schedule the diagnostic test; and
transmitting a confirmation of the scheduled diagnostic test to the user over the network.

21.-22. (canceled)

23. A handheld device for facilitating the treatment of a medical condition in a subject, the handheld device comprising:
an interactive diagnostic support system comprising a platform comprising a processor and memory, both of which are coupled with at least one server, the at least one server in operative communication with a network, accessible by at least one user via one or more clients, comprising at least one application executable by the processor, configured to interact with data stored at least within the memory of the platform, the platform configured to:
display via a user interface of the handheld device a list of inquiries for distinguishing between a plurality of medical conditions,
receive, on the server, data from a user in response to the list of inquiries,
access and compare the received data from the user with medical reference data stored at least partially within the memory of the platform to identify a subset of medical conditions that correlate with the received data,
generate an updated list of inquiries to distinguish between the medical conditions of the identified subset, and
display via the user interface the subset of medical conditions and the updated list of inquiries;
wherein the received data is associated with a patient and comprises key indicators and, as relevant, pathognomonic data.

24. The handheld device of claim 23, wherein the platform is further configured to identify and display on the handheld device one or more diagnostic tests, the results of which would be useful in distinguishing between the medical conditions of the identified subset.

25. The handheld device of claim 24, wherein the server of the platform is in operative communication with one or more laboratories over the network and the platform is further configured to interact with the one or more laboratories in response to a request from the user to schedule a diagnostic test.

26. (canceled)

27. The handheld device of claim 23, wherein the plurality of medical conditions comprise general medicine and pediatric conditions and are selected from a group consisting conditions characterized by deficiency of immune function or regulation, autoimmune diseases, and auto-inflammatory diseases.

28.-30. (canceled)

31. The handheld device of claim 23, wherein the medical reference data stored at least partially within the memory of the platform is updatable in real time via multiple users using the one or more clients over the network.

32. The handheld device of claim 23, wherein the medical reference data comprises a plurality of medical conditions, with one or more phenotypic manifestations, characteristics, molecular causes, and categories assigned to each medical condition.

33.-34. (canceled)

35. A handheld device comprising:
a processor and memory;
a first interactive educational and treatment application executable by the processor of the handheld device, the first application configured to interact with a networked platform comprising a processor and memory, both of which are coupled with at least one server, the at least one server in operative communication with a network, accessible by at least one user via one or more clients, and comprising at least one second application executable by the processor of the platform and capable of interacting with data stored at least partially in the memory of the platform;
at least one user interface configured to display a list of available data sets of the data stored at least partially in the memory of the platform, each data set associated with a medical condition and receive input data from a user comprising key indicators pathognomonic data a subject;
wherein the first application is further executable to transmit the input data to the networked platform and the second application is further executable to correlate the input data received with the list of available data sets of the networked platform and identify a first data set comprising a medical condition associated with the input data received.

36. (canceled)

37. The handheld device of claim 35, wherein the data stored at least partially in the memory of the platform is stored in a reference database and the reference database is updatable in real time via multiple users over the network.

38. (canceled)
